(19) United States
(12) Patent Application Publication
(10) Pub. No.: US 2018/0147234 A1
(43) Pub. Date: May 31, 2018

(54) COMPOSITIONS AND METHODS FOR TREATMENT OF FIBROMYALGIA

(71) Applicant: Luitpold Pharmaceuticals, Inc., Shirley, NY (US)

(72) Inventors: Erin Michelle Malloy, Malvern, PA (US); Todd Koch, Harleysville, PA (US); Marc L. Tokars, Douglassville, PA (US)

(21) Appl. No.: 15/577,267

(22) PCT Filed: May 27, 2016
(86) PCT No.: PCT/US16/34608

§ 371 Date: Nov. 27, 2017

Related U.S. Application Data
(60) Provisional application No. 62/168,072, filed on May 29, 2015.

(51) Int. Cl. A61K 31/7135; A61P 25/00

(52) U.S. Cl. CPC A61K 31/7135; A61P 25/00

(57) ABSTRACT

Provided herein are methods and compositions for the treatment of Fibromyalgia (FM) or Fibromyalgia syndrome (FMS). One aspect provides treating a subject having, suspected of having, or diagnosed with FM or FMS with a pharmaceutically effective amount of an iron carbohydrate complex.

### CROSS-REFERENCE TO RELATED APPLICATIONS

The present application claims the benefit of U.S. Provisional Application Ser. No. 62/168,072 filed 29 May 2015, which is incorporated herein by reference in its entirety.

### BACKGROUND OF THE INVENTION

Treatment of Fibromyalgia (FM) or Fibromyalgia syndrome (FMS) has historically consisted of treating a cluster of symptoms. Symptoms of FM or FMS can include pain, insomnia, restless leg syndrome, urinary frequency, or irritable bowel syndrome. The central symptom of FM or FMS, namely widespread pain, is presently thought to result from neuro-chemical imbalances including activation of inflammatory pathways in the brain which results in abnormalities in pain processing.

Several drugs have been approved for the systematic treatment of FM or FMS, such as Lyrica® (pregablin), Cymbalta® (duloxetine), or Savella® (milnacipran).

Fibromyalgia is a chronic, wide-spread myalgia that occurs in about 3.5% of females and about 0.5% of males in the United States. Co-occurring with the myalgia, other common symptoms are sleep disturbance, fatigue, headache, morning stiffness, irritable bowel syndrome (IBS), interstitial cystitis (IC), dyspareunia, or mood disturbance. The cause of fibromyalgia is currently unknown.

### SUMMARY OF THE INVENTION

Among the various aspects of the present disclosure is the provision of an iron carbohydrate complex for the treatment of Fibromyalgia.

Another aspect provides for methods of treating a subject with Fibromyalgia (FM) or Fibromyalgia syndrome (FMS), including administering an iron carbohydrate complex to a subject in need thereof. The iron carbohydrate complex can be selected from the group consisting of an iron carboxymaltose complex, iron sucrose complex, an iron mannitol complex, an iron polyisomaltose complex, an iron polymaltose complex, an iron gluconate complex, an iron sorbitol complex, an iron polyglucose sorbitol carboxymethyl ether complex, an iron polyglucose sorbitol, iron carboxymethyl ether complex, or an iron hydrogenated dextran complex.

Method features discussed above can be combined with other features discussed below.

In some embodiments, the iron carbohydrate complex has a substantially non-immunogenic carbohydrate component. In some embodiments, the iron carbohydrate complex includes iron carboxymaltose complex, an iron polyglucose sorbitol carboxymethyl ether complex, or an iron polyisomaltose complex. In some embodiments, the iron carboxymaltose complex comprises VIT-45. In some embodiments, iron polyglucose sorbitol carboxymethyl ether complex comprises Ferumoxytol. In some embodiments, the iron polyisomaltose comprises Monofer. Features related to microspheres can be combined with other features discussed above and below.

In some embodiments, administration of the iron carbohydrate complex is administered in an amount effective to inhibit, slow, limit, remove, or prevent symptoms associated with Fibromyalgia (FM) or Fibromyalgia syndrome (FMS). In some embodiments, administration of the iron carbohydrate complex substantially inhibits, slows, limits, removes, or prevents one or more of: chronic widespread pain, painful response to pressure, painful response to tactile pressure (allodynia), fatigue, headache, debilitating fatigue, sleep disturbance, joint stiffness, morning stiffness, difficulty with swallowing, bowel abnormalities, bladder abnormalities, numbness, tingling, tingling of the skin (paresthesias), prolonged muscle spasms, weakness in the limbs, nerve pain, muscle twitching, palpitations, functional bowel disturbances, irritable bowel syndrome (IBS), cognitive dysfunction, depression, anxiety, stress-related disorders, interstitial cystitis (IC), dyspareunia, or mood disturbance.

Features related to the combination of microspheres and matrix material can be combined with other features discussed above and below.

In some embodiments, the administration of the iron carbohydrate complex substantially improves one or more of: FIQR total score; Brief Pain Inventory, Pain Severity, and Pain Interference scores; Fatigue Visual Numeric Scale; and iron indices. In some embodiments, the administration of the iron carbohydrate complex substantially reduces Revised Fibromyalgia Impact Questionnaire (FIQR) value, reduces International Restless Legs Syndrome (IRLS) value, reduces Brief Pain Inventory (BPI) value, reduces Pain Severity value, reduces Pain Interference value, reduces Fatigue Visual Numeric value, reduces required intervention for fibromyalgia, reduces an amount of time to fibromyalgia intervention, or reduces proportion of relapse, or any combination thereof when compared to baseline values.

Features related to the combination of microspheres and matrix material can be combined with other features discussed above and below.

In some embodiments, the iron carbohydrate complex is administered at a single dosage unit of at least about 0.1 grams of elemental iron. In some embodiments, the iron carbohydrate complex is administered at a single dosage unit of at least about 0.1 grams, at least about 0.2 grams, at least about 0.3 grams, at least about 0.4 grams, at least about 0.5 grams, 0.6 grams, at least about 0.7 grams; at least about 0.8 grams; at least about 0.9 grams; at least about 1.0 grams; at least about 1.1 grams; at least about 1.2 grams; at least about 1.3 grams; at least about 1.4 grams; at least about 1.5 grams; at least about 1.6 grams; at least about 1.7 grams; at least about 1.8 grams; at least about 1.9 grams; at least about 2.0 grams; at least about 2.1 grams; at least about 2.2 grams; at least about 2.3 grams; at least about 2.4 grams; or at least about 2.5 grams of elemental iron. In some embodiments, the iron carbohydrate complex is administered at a single dosage unit of about 0.1 grams, about 0.2 grams, about 0.3 grams, about 0.4 grams, about 0.5 grams, 0.6 grams, about 0.7 grams; about 0.8 grams; about 0.9 grams; about 1.0 grams; about 1.1 grams; about 1.2 grams; about 1.3 grams; about 1.4 grams; about 1.5 grams; about 1.6 grams; about 1.7 grams; about 1.8 grams; about 1.9 grams; about 2.0 grams; about 2.1 grams; about 2.2 grams; about 2.3 grams; about 2.4 grams; or about 2.5 grams of elemental iron. In some embodiments, the iron carbohydrate complex is administered at a single dosage unit of up to about 0.1 grams, up to about 0.2 grams, up to about 0.3 grams, up to about 0.4 grams, up to about 0.5 grams, 0.6 grams, up to about 0.7 grams; up to about 0.8 grams; up to about 0.9 grams; up to about 1.0 grams; up to about 1.1 grams; up to about 1.2 grams; up to about 1.3 grams; up to about 1.4 grams; up to about 1.5 grams; up to about 1.6 grams; up to about 1.7 grams; up to about 1.8 grams; up to about 1.9 grams; up to about 2.0 grams; up to about 2.1 grams; up to about 2.2 grams; up to about 2.3 grams; up to about 2.4 grams; or up to about 2.5 grams of elemental iron.

Features related to the combination of microspheres and matrix material can be combined with other features discussed above and below.

In some embodiments, the iron carbohydrate complex is administered in about 15 minutes or less. In some embodiments, the iron carbohydrate complex is administered in about 14 minutes or less, about 13 minutes or less, about 12 minutes or less, about 11 minutes or less, about 10 minutes or less, about 9 minutes or less, about 8 minutes or less, about 7 minutes or less, about 6 minutes or less, about 5 minutes or less, about 4 minutes or less, about 3 minutes or less, or about 2 minutes or less.

Features related to the combination of microspheres and matrix material can be combined with other features discussed above and below.

In some embodiments, administering further includes administering pregablin, duloxetine, or milnacipran.

Features related to the combination of microspheres and matrix material can be combined with other features discussed above and below.

In some embodiments, the amount of time after administration of the iron carbohydrate complex symptoms associated with Fibromyalgia (FM) or Fibromyalgia syndrome (FMS) are substantially inhibited, slowed, limited, removed, or prevented is between about 0 days and about 50 days. In some embodiments, the amount of time after administration of the iron carbohydrate complex symptoms associated with Fibromyalgia (FM) or Fibromyalgia syndrome (FMS) are substantially inhibited, slowed, limited, removed, or prevented is about 14 days, about 28, or about 42 days.

Features related to the combination of microspheres and matrix material can be combined with other features discussed above and below.

Other objects and features will be in part apparent and in part pointed out hereinafter.

### DETAILED DESCRIPTION OF THE INVENTION

The present disclosure is based, at least in part, on the discovery that intravenous iron carbohydrate complexes can treat fibromyalgia. As shown herein, treating subjects diagnosed with fibromyalgia with intravenous iron carbohydrate complexes statistically significantly greater mean improvement was observed for Injectafer as compared to placebo for FIQR total score, Brief Pain Inventory Pain Severity and Pain Interference scores, Fatigue Visual Numeric Scale, iron indices, or hemoglobin.

As shown herein, treating subjects diagnosed with fibromyalgia with intravenous iron carbohydrate complexes can improve quality of life. Treatment of subjects diagnosed with fibromyalgia can have improved pharmacoeconomic benefits compared to standard treatments. Subjects diagnosed with fibromyalgia having intravenous iron carbohydrate treatment can have less dependence on opioid medications or can have reduction in missed time from work.

An iron carbohydrate complex can be administered parenterally at relatively high single unit dosages, thereby providing a safe and efficient means for delivery of a total dose of iron in fewer sessions over the course of therapeutic treatment. Benefits to a subject receiving an infusion/injection of IV iron vs. daily pills and doctors visits include not requiring multiple prescriptions for multiple symptoms.

The side effects of IV iron can be more transient and thus can be preferable to traditional fibromyalgia medications. Side effects of many fibromyalgia drugs can include weight gain, edema of hands or feet, or drowsiness. Existing fibromyalgia drugs can be expensive if the patient does not have insurance and is paying out of pocket.

Subjects diagnosed with fibromyalgia can have a high occurrence of low iron levels. Conventional iron replacement which includes oral administration of iron, blood transfusion, or both, can have issues. These issues can include possible decreased efficacy of orally administered iron secondary to adverse side effects (for example nausea, constipation) leading to noncompliance. Blood transfusions often remain a last resort for multiple reasons including patient choice, risk of transmission of known or unknown pathogens, immunological impact, or transfusion reaction(s). Moreover, replenishment of depletion iron reserves can take months to occur by using oral substitution. While most patients can tolerate oral iron without difficulty, up to 40% can have symptoms attributable to oral iron replacement therapy. Side effects can often occur within an hour after ingestion and may be mild, but also can be more serious, with pain, vomiting, diarrhea, or constipation.

Administration of intravenous iron can stimulate a natural response to endogenous erythropoietin to achieve normal hemoglobin levels. Thus, iron therapy in any form is generally unable to generate hemoglobin levels above what is physically normal for the patient, and therefore cannot generate “excessive” hemoglobin levels. With intravenous iron, excess iron (i.e., that which is not needed to achieve a hemoglobin normal for that individual) can be stored and released slowly as needed for erythropoiesis.

Iron Carbohydrate Complex

Iron carbohydrate complexes are commercially available, or have well known syntheses. Examples of iron carbohydrate complexes include iron monosaccharide complexes, iron disaccharide complexes, iron oligosaccharide complexes, or iron polysaccharide complexes, such as: iron carboxymaltose, iron sucrose, iron polyisomaltose, iron polymaltose, iron gluconate, iron sorbitol, iron hydrogenated dextran, which may be further complexed with other compounds, such as sorbitol, citric acid or gluconic acid (for example iron dextrin-sorbitol-citric acid complex or iron sucrose-gluconic acid complex), or mixtures thereof.

Intravenous iron agents can be colloidal dispersions consisting of particles (e.g., spheroid) with a carbohydrate shell and ferric oxyhydroxide cores. Therapeutic efficacy of intravenous iron agents can depend on uptake of the circulating agent by macrophages of the reticuloendothelial system (RES) and enterocytes. Iron can then be mobilized slowly from the engulfed particles and released over days, weeks, or months from the macrophage to bind to circulating transferrin. Utilization of iron dose can be most rapid and complete in patients with severe iron deficiency. Because intravenous iron agents can share the same fundamental chemistry or depend on RES uptake of iron agent prior to delivery of iron to erythroid marrow, the agents can be distinguished in clinical practice by the rate that a maximum single dose can be safely administered or the individual adverse events profile associated with administration of the iron agent.

Applicants have discovered that certain characteristics of iron carbohydrate complexes make them amenable to administration at dosages far higher than contemplated by current administration protocols. Preferably, iron carbohydrate complexes for use in the methods described herein are those which have one or more of the following characteristics: a nearly neutral pH (e.g., about 5 to about 7); physiological osmolarity; stable carbohydrate component; an iron core size no greater than about 9 nm; mean diameter particle size no greater than about 35 nm, preferably about 25 nm to about 30 nm; slow or competitive delivery of the complexed iron to endogenous iron binding sites; serum half-life of over about 7 hours; low toxicity; non-immunogenic carbohydrate component; or low risk of anaphylactoid/hypersensitivity reactions.

Some iron carbohydrate complexes can act as a hapten, which can bind an antibody (e.g., a Dextran antibody) without inducing anaphylaxis or an immune response. So while there may be a reaction to a Dextran antibody, there may not be anaphylaxis or an immungenic response. The ability to administer relatively high doses of iron carbohydrate complexes described herein can arise, at least in part, from reduced immunogenic potential or absence of dextran-induced anaphylactic reactions.

It is within the skill of the art to test various characteristics of iron carbohydrate complexes as so determine amenability to use in the methods described herein. For example, pH or osmolarity can be straightforward determinations performed on a sample formulation. Likewise, techniques such as electron micrograph imaging, transmission electron microscopy, or atomic force microscopy provide direct methods to analyze both iron core or particle size (see U.S. Pat. No. 7,754,702; U.S. Pat. No. 8,431,549; or U.S. Pat. No. 8,895,612, each incorporated herein by reference).

The stability of an iron carbohydrate complex can be assessed through physicochemical properties such as kinetic characteristics, thermodynamic characteristics, or degradation kinetics. Useful techniques to assess physical or electronic properties include absorption spectroscopy, X-ray diffraction analysis, transmission electron microscopy, atomic force microscopy, or elemental analysis. See Kudasheva 2004 J Inorg Biochem 98, 1757-1769. Pharmacokinetics can be assessed, for example, by iron tracer experiments. Hypersensitivity reactions can be monitored or assessed as described in, for example, Bailie 2005 Nephrol Dial Transplant, 20(7), 1443-1449. Safety, efficacy, or toxicity in human subjects can be assessed, for example, as described in Spinowitz 2005 Kidney Intl 68, 1801-1807.

An iron carbohydrate complex can have a pH between 5.0-7.0; physiological osmolarity; an iron core size no greater than 9 nm; mean diameter particle size no greater than 30 nm; serum half-life of over 10 hours; or a non-immunogenic carbohydrate component. One example of an iron carbohydrate complex for use in the methods described herein is an iron carboxy-maltose complex (e.g., polynuclear iron (III)-hydroxide 4(R)-(poly-(1→4)-O-α-glucopyranosyl)-oxy-2(R),3(S),5(R),6-tetrahydroxy-hexanoate, “VIT-45”, or “Injectafer®”). Another example of an iron carbohydrate complex for use in the methods described herein is a carboxyalkylated reduced polysaccharide iron oxide complex (e.g., Ferumoxytol, described in U.S. Pat. No. 6,599,498). Another example of an iron carbohydrate complex for use in the methods described herein is Monofer (iron isomaltoside 1000), an iron polyisomaltose.

An iron carbohydrate complex, for use in methods disclosed herein, can contain about 24% to about 32% elemental iron, or about 28% elemental iron. An iron carbohydrate complex, for use in methods disclosed herein, can contain about 25% to about 50% carbohydrate (e.g., total glucose). An iron carbohydrate complex, for use in methods disclosed herein, can be about 90,000 Daltons to about 800,000 Daltons, more preferably 100,000 Daltons to about 350,000 Daltons.

Iron Carboxymaltose Complex (e.g., Injectafer®, VIT-45)

An iron carbohydrate complex for use in the methods described herein can be an iron carboxymaltose complex. An example of an iron carboxymaltose complex is polynuclear iron (III)-hydroxide 4(R)-(poly-(1→4)-O-α-glucopyranosyl)-oxy-2(R),3(S),5(R),6-tetrahydroxy-hexanoate (“VIT-45”, or “Injectafer®”). VIT-45 is a stable Type I polynuclear iron (III) hydroxide carbohydrate complex that can be administered as parenteral iron therapy for the treatment of FM or FMS. VIT-45 can be represented by the chemical formula: [FeOx(OH)y(H2O)z]n [{(C6H10O5)m (C6H12O7)}l]k, where n is about 103, m is about 8, l is about 11, and k is about 4). The molecular weight of VIT-45 is about 150,000 Da.

The degradation rate or physicochemical characteristics of the iron carbohydrate complex can make it an efficient means of parenteral iron delivery. It can be more efficient or less toxic than the lower molecular weight complexes, such as iron sorbitol/citrate complex, and does not have the same limitations of high pH or osmolarity that leads to dosage or administration rate limitations in the case of, for example, iron sucrose or iron gluconate.

The iron carboxymaltose complex generally does not contain dextran or does not raise an immuogenic response; therefore, the risk of anaphylactoid/hypersensitivity reactions can be very low compared to iron dextran. While some residual reaction to dextran antibodies may occur, such reaction does not raise an immunogenic reaction in a subject. The iron carboxymaltose complex can have a nearly neutral pH (5.0 to 7.0) and physiological osmolarity, which makes it possible to administer higher single unit doses over shorter time periods than other iron-carbohydrate complexes. The iron carboxymaltose complex can mimic physiologically occurring ferritin. The carbohydrate moiety of iron carboxymaltose complex can be metabolized by the glycolytic pathway. Like iron dextran, the iron carboxymaltose complex can be more stable than iron gluconate or sucrose. The iron carboxymaltose complex can produce a slow or competitive delivery of the complexed iron to endogenous iron binding sites resulting in an acute toxicity one-fifth that of iron sucrose. These characteristics of the iron carboxymaltose complex allow administration of higher single unit doses over shorter periods of time than, for example, iron gluconate or iron sucrose. Higher single unit doses can result in the need for fewer injections to replete iron stores, and consequently can be often better suited for outpatient use.

After intravenous administration, the iron carboxymaltose complex can be found in the liver, spleen, or bone marrow. Pharmacokinetic studies using positron emission tomography have demonstrated a fast initial elimination of radioactively labeled iron (Fe) 52Fe/59Fe VIT-45 from the blood, with rapid transfer to the bone marrow or rapid deposition in the liver or spleen (see e.g., Beshara 2003 Br J Haematol 120(5), 853-859). Eight hours after administration, 5 to 20% of the injected amount was observed to be still in the blood, compared with 2 to 13% for iron sucrose. The projected calculated terminal half-life (t½) was approximately 16 hours, compared to 3 to 4 days for iron dextran or 6 hours for iron sucrose. Other studies (maximal total serum iron concentration was approximately 150 μg/mL or 320 μg/mL following 500 mg or 1000 mg doses, respectively) showed VIT-45 has a monoexponential elimination pattern with a t½ in the range 7 to 18 hours, with negligible renal elimination.

The iron in the iron carboxymaltose complex slowly dissociates from the complex and can be efficiently used in the bone marrow for hemoglobin (Hgb) synthesis. Under VIT-45 administration, red cell utilization, followed for 4 weeks, ranged from 61% to 99%. Despite the relatively higher uptake by the bone marrow, experiments show there was no saturation of marrow transport systems. In addition, the reticuloendothelial uptake of this complex reflects the safety of polysaccharide complexes. Non-saturation of transport systems to the bone marrow indicated the presence of a large interstitial transport pool (e.g., transferrin).

Single-dose toxicity studies have demonstrated safety and tolerance in rodents and dogs of intravenous doses of an iron carboxymaltose complex (VIT-45) up to 60 times more than the equivalent of an intravenous infusion of 1,000 mg iron once weekly in human subjects. Pre-clinical studies in dogs and rats administered VIT-45 in cumulative doses up to 117 mg iron/kg body weight over 13 weeks showed no observed adverse effect level in dose-related clinical signs of iron accumulation in the liver, spleen, or kidneys. No treatment-related local tissue irritation was observed in intra-arterial, perivenous, or intravenous tolerance studies in the rabbit. In vitro and in vivo mutagenicity tests provided no evidence that VIT-45 is clastogenic, mutagenic, or causes chromosomal damage or bone marrow cell toxicity. There were no specific responses to VIT-45 in a dextran antigenicity test.

Thousands of subjects have been treated with an iron carboxymaltose complex (VIT-45) in open label clinical trials. Many of these subjects have received at least one dose of 15 mg/kg (up to a maximum dose of 1,000 mg) of VIT-45 over 15 minutes intravenously. Few adverse events and no serious adverse events or withdrawals due to adverse events related to VIT-45 administration have been reported. No clinically relevant adverse changes in safety laboratories have been seen.

The physicochemical characteristics of the iron carboxymaltose complex (e.g., VIT-45), the pattern of iron deposition, and the results of the above described studies demonstrate that iron carboxymaltose complex can be safely administered at high single unit therapeutic doses as described herein.

Prior to the approval of non-dextran formulations, the risk of systemic adverse reactions restricted use of various parenteral iron preparations. Iron carboxymaltose (e.g., Injectafer®) can offer significant advantages compared to other available intravenous iron preparations.

Iron dextran, the first parenteral iron product available in the US, has been associated with an incidence of anaphylaxis/anaphylactoid reactions (i.e., dyspnea, wheezing, hypotension, urticaria, angioedema) as high as 1.7%. Over the last 20 years, 30 deaths have been attributed to the use of IV iron dextran. The high incidence of anaphylaxis/anaphylactoid reactions is believed to be caused by the formation of antibodies to the dextran moiety. Although some have suggested that high molecular weight (HMW) iron dextran is associated with a higher rate of life threatening adverse events or anaphylactic reactions in comparison to low molecular weight (LMW) iron dextran, the US Food and Drug Administration was unable to find a clear difference after an examination of post-marketing data, clinical trial data, death certificates, or emergency room diagnoses. Iron dextran is limited to second line therapy for treatment of iron deficiency.

More recently approved, non-dextran intravenous irons such as iron sucrose or iron gluconate, do not contain the dextran moiety, but they can have significant dosage or administration rate limitations. If the body's ability to handle (i.e., sequester, store, or transport) iron is overwhelmed, a reaction to excess free iron referred to as a bioactive iron reaction can occur. These IV iron compounds carry a significant risk of bioactive iron reactions at higher doses. These reactions are characterized by hypotension (without allergic signs) accompanied by pain in the chest, abdomen, flank, nausea, vomiting, or diarrhea. But these iron agents are not FDA approved for the treatment of iron deficiency in non-chronic kidney disease populations.

Due to its structure, iron carboxymaltose (e.g., Injectafer®) can be more stable than iron gluconate or iron sucrose, producing a slow delivery of the complexed iron to endogenous iron binding sites. Further, iron carboxymaltose can have an acute toxicity in animals approximately ⅕ that of iron sucrose. These characteristics of iron carboxymaltose (e.g., Injectafer®) can make it possible to administer much higher single doses over shorter periods of time than iron gluconate or iron sucrose, resulting in the need for fewer administrations to replenish iron stores, consequently making it better suited for outpatient use.

Iron carboxymaltose received Medicines and Healthcare products Regulatory Agency (MHRA) approval on 15 Jun. 2007 for the use of Injectafer® (EU Trade name: Ferinject) in 18 EU (European Union) countries and later in Switzerland. Ferric carboxymaltose was first approved as a prescription only medicine on 6 Jul. 2007 in The Netherlands. At present, Injectafer® has received regulatory approval for marketing authorization in 470 countries and is currently marketed in 55 of the countries. Countries with marketing authorization include Argentina, Australia, Austria, Bangladesh, Belgium, Bolivia, Brazil, Bulgaria, Chile, Colombia, Costa Rica, Croatia, Cyprus, Czech Republic, Denmark, Ecuador, El Salvador, Estonia, Finland, France, Germany, Greece, Guatemala, Honduras, Hungary, Iceland, India, Iran, Ireland, Israel, Italy, Jordan, Kazakhstan, Kuwait, Latvia, Lebanon, Liechtenstein, Lithuania, Luxembourg, Malta, The Netherlands, New Zealand, Norway, Pakistan, Peru, Poland, Portugal, Romania, Russia, Serbia, Singapore, Slovak Republic, South Korea, Slovenia, South Africa, Spain, Sweden, Switzerland, Turkey, Ukraine, United Kingdom, and the United States.

Polyglucose Sorbitol Carboxymethyl Ether-Coated Non-Stoichiometric Magnetite (e.g., Ferumoxytol)

An iron carbohydrate complex for use in the methods described herein can be a polyglucose sorbitol carboxymethyl ether-coated non-stoichiometric magnetite (e.g., “ferumoxytol”) (see Spinowitz 2005 Kidney Intl 68, 1801-1807). Ferumoxytol is understood to be a superparamagnetic iron oxide that is coated with a low molecular weight semi-synthetic carbohydrate, polyglucose sorbitol carboxymethyl ether. Ferumoxytol and its synthesis are described in U.S. Pat. No. 6,599,498, incorporated herein by reference. Safety, efficacy, and pharmacokinetics of ferumoxytol are as described, for example, in Landry 2005 Am J Nephrol 25, 400-410, 408; and Spinowitz 2005 Kidney Intl 68, 1801-1807.

The iron oxide of ferumoxytol is understood to be a superparamagnetic form of non-stoichiometric magnetite with a crystal size of about 6.2 to 7.3 nm. Average colloidal particle size can be about 30 nm, as determined by light scattering. Molecular weight is approximately 750 kD. The osmolarity of ferumoxytol is isotonic at 297 mOsm/kg and the pH is neutral. The blood half-life of ferumoxytol can be approximately 10-14 hours. It has been previously reported that ferumoxytol can be given by direct intravenous push over 1-5 minutes in doses up to 1,800 mg elemental iron per minute, with maximal total dose up to 420 mg per injection (see Landry 2005 Am J Nephrol 25, 400-410, 408).

Iron Polyisomaltose (e.g., Monofer).

An iron carbohydrate complex for use in the methods described herein can be an iron polyisomaltose, such as Monofer (generically named iron isomaltoside 1000). An iron polyisomaltose is understood as a type of iron carbohydrate complex that includes isomaltose units in the carbohydrate component. An isomaltose is a disaccharide similar to maltose, but with a α-(1-6)-linkage between two glucose units instead of an α-(1-4)-linkage. One example of an iron polyisomaltose complex is an iron isomaltoside (e.g., Monofer®), where the carbohydrate component is a pure linear chemical structure of repeating α1-6 linked glucose units. In contrast, a dextran is a branched glucan with straight chains having α1-6 glycosidic linkages and branches beginning from α1-3 linkages. Physiochemical properties of the iron isomaltoside Monofer® are described in Jahn 2011 Eur J Pharma and Biopharma 78, 480-49.

The iron isomaltoside Monofer® (i.e., one example of an iron polyisomaltose complex) can avoid dextran-induced anaphylactic reactions or reduce immunogenicity compared to dextran (Jahn 2011 Eur J Pharma and Biopharma 78, 480-49).

Therapeutic Methods

Also provided is a process of treating Fibromyalgia (FM) or Fibromyalgia syndrome (FMS) in a subject in need of administration of a therapeutically effective amount of an iron carbohydrate complex, so as to ameliorate, decrease, eliminate, or prevent one or more symptoms of FM or FMS.

Generally, treating FM or FMS can include preventing or delaying the appearance of one or more clinical symptoms in a subject that may be afflicted with or predisposed to the state, disease, disorder, or condition but does not yet experience or display clinical or subclinical symptoms thereof. Treating can also include inhibiting FM or FMS, e.g., arresting or reducing the development of the disease or at least one clinical or subclinical symptom thereof. Furthermore, treating can include relieving FM or FMS, e.g., causing regression of FM or FMS or at least one clinical or subclinical symptoms. The benefit to a subject to be treated can be either statistically significant or at least perceptible to the subject or to the physician.

FM or FMS can be characterized by chronic widespread pain or a heightened or painful response to pressure. Because fibromyalgia symptoms are not restricted to pain, the alternative term fibromyalgia syndrome (FMS) can be used for the condition. Other FM symptoms include debilitating fatigue, sleep disturbance, or joint stiffness. Some subjects with FM also can have difficulty with swallowing, bowel or bladder abnormalities, numbness or tingling, or cognitive dysfunction. Many subjects experience cognitive dysfunction (known as “fibrofog”), which may be characterized by impaired concentration, problems with short or long-term memory, short-term memory consolidation, impaired speed of performance, inability to multi-task, cognitive overload, or diminished attention span. Fibromyalgia can be often associated with anxiety or depressive symptoms Fibromyalgia can be frequently associated with psychiatric conditions such as depression, anxiety, or stress-related disorders such as posttraumatic stress disorder. Not all subjects with fibromyalgia experience all associated symptoms. Administration of an iron carbohydrate complex can substantially inhibit, slow, limit, remove, or prevent one or more of the above described symptoms.

It is thought that a fibromyalgia subject may have a lower threshold for pain because of increased reactivity of pain-sensitive nerve cells in the spinal cord or brain. The pain in fibromyalgia may result primarily from pain processing pathways functioning abnormally, i.e., the volume of neurons being set too high and this hyper-excitability of pain processing pathways or under-activity of inhibitory pain pathways in the brain results in the affected subject experiencing pain. Some neurochemical abnormalities that occur in fibromyalgia also regulate mood, sleep or energy, thus explaining why mood, sleep or fatigue problems are commonly co-morbid with fibromyalgia.

Methods described herein are generally performed on a subject in need thereof. A subject in need of the therapeutic methods described herein can be a subject having, diagnosed with, suspected of having, or at risk for developing FM or FMS. A determination of the need for treatment will typically be assessed by a history or physical exam consistent with the disease or condition at issue. Diagnosis of the various conditions treatable by the methods described herein is within the skill of the art. Diagnosis of FM or FMS can be according to the ACR 1990 criteria: (i) a history of widespread pain lasting more than three months, affecting all four quadrants of the body, i.e., both sides, and above and below the waist; and (ii) pain in multiple designated “tender points”. Diagnosis of FM or FMS can be according to the widespread pain index (WPI) and symptom severity scale (SS), where WPI counts up to 19 general body areas in which a subject has experienced pain in the preceding two weeks, SS rates the severity of the person's fatigue, unrefreshed waking, cognitive symptoms, or general somatic symptoms, each on a scale from 0 to 3, for a composite score ranging from 0 to 12. The revised criteria for diagnosis can be: WPI≥7 and SS≥5 OR WPI 3-6 and SS≥9; symptoms have been present at a similar level for at least three months, and no other diagnosable disorder otherwise explains the pain.

The iron carbohydrate complexes can be administered in an amount effective to inhibit, slow, limit, remove, or prevent symptoms associated with Fibromyalgia. For example, symptoms can include chronic widespread pain, painful response to pressure, painful response to tactile pressure (allodynia), fatigue, debilitating fatigue, sleep disturbance, joint stiffness, difficulty with swallowing, bowel abnormalities, bladder abnormalities, numbness, tingling, tingling of the skin (paresthesias), prolonged muscle spasms, weakness in the limbs, nerve pain, muscle twitching, palpitations, functional bowel disturbances, cognitive dysfunction, depression, anxiety, or stress-related disorders. As another example, symptoms can include sleep disturbance, fatigue, headache, morning stiffness, irritable bowel syndrome (IBS), interstitial cystitis (IC), dyspareunia, or mood disturbance. As another example, symptoms can include symptoms in Gerwin, 2005.

The subject can be an animal subject, including a mammal, such as horses, cows, dogs, cats, sheep, pigs, mice, rats, monkeys, hamsters, guinea pigs, or chickens, or humans. For example, the subject can be a human subject.

Generally, a safe and effective amount of an iron carbohydrate complex is, for example, an amount that would cause the desired therapeutic effect in a subject while minimizing undesired side effects. In various embodiments, an effective amount of an iron carbohydrate complex described herein can substantially inhibit, slow, limit, remove, or prevent one or more of chronic widespread pain, painful response to pressure, painful response to tactile pressure (allodynia), fatigue, debilitating fatigue, sleep disturbance, joint stiffness, difficulty with swallowing, bowel abnormalities, bladder abnormalities, numbness, tingling, tingling of the skin (paresthesias), prolonged muscle spasms, weakness in the limbs, nerve pain, muscle twitching, palpitations, functional bowel disturbances, cognitive dysfunction, depression, anxiety, or stress-related disorders such as posttraumatic stress disorder. In various embodiments, an effective amount of an iron carbohydrate complex described herein can substantially inhibit, slow, limit, remove, or prevent symptoms associated with fibromyalgia. For examples, symptoms associated with fibromyalgia can include chronic widespread pain, painful response to pressure, painful response to tactile pressure (allodynia), fatigue, headache, debilitating fatigue, sleep disturbance, joint stiffness, morning stiffness, difficulty with swallowing, bowel abnormalities, bladder abnormalities, numbness, tingling, tingling of the skin (paresthesias), prolonged muscle spasms, weakness in the limbs, nerve pain, muscle twitching, palpitations, functional bowel disturbances, irritable bowel syndrome (IBS), cognitive dysfunction, depression, anxiety, stress-related disorders, interstitial cystitis (IC), dyspareunia, or mood disturbance. In various embodiments, an effective amount of an iron carbohydrate complex described herein can substantially reduce Revised Fibromyalgia Impact Questionnaire (FIQR) value, reduce International Restless Legs Syndrome (IRLS) value, reduce Brief Pain Inventory (BPI) value, reduce Pain Severity value, reduce Pain Interference value, reduce Fatigue Visual Numeric value, reduce required intervention for fibromyalgia, reduce an amount of time to fibromyalgia intervention, or reduce proportion of relapse.

For example, minimized undesirable side effects can include those related to hypersensitivity reactions, sometimes classified as sudden onset closely related to the time of dosing, including hypotension, bronchospasm, laryngospasm, angioedema or uticaria or several of these together. Hypersensitivity reactions are reported with all current intravenous iron products independent of dose (see generally Bailie 2005 Nephrol Dial Transplant, 20(7), 1443-1449). As another example, minimized undesirable side effects can include those related to labile iron reactions, sometimes classified as nausea, vomiting, cramps, back pain, chest pain, or hypotension. Labile iron reactions are more common with iron sucrose, iron gluconate, or iron dextran when doses are large or given fast. Generally, treatment-emergent adverse events may occur in less than about 5% of treated subjects. For example, treatment-emergent adverse events may occur in less than 4% or 3% of treated subjects. As another example, treatment-emergent adverse events may occur in less than about 2% of treated patients.

Generally, the amount of time for an iron carbohydrate complex to substantially inhibit, slow, limit, remove, or prevent symptoms associated with fibromyalgia can be about 0 to about 50 days. For example, the amount of time can be about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, or about 50 days after administration of the iron carbohydrate complex.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an iron carbohydrate complex can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to ameliorate, decrease, eliminate, or prevent on or more symptoms associated with FM or FMS.

The amount of a composition described herein that can be combined with a pharmaceutically acceptable carrier to produce a single dosage form will vary depending upon the host treated or the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity or therapeutic efficacy of compositions described herein can be determined by standard pharmaceutical procedures in cell cultures or experimental animals for determining the LD₅₀ (the dose lethal to 50% of the population) or the ED₅₀, (the dose therapeutically effective in 50% of the population). The dose ratio between toxic and therapeutic effects can be the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated or the severity of the disorder; activity of the specific compound employed; the specific composition employed; the age, body weight, general health, sex or diet of the subject; the time of administration; the route of administration; the rate of excretion of the composition employed; the duration of the treatment; drugs used in combination or coincidental with the specific compound employed; or like factors well known in the medical arts (see e.g., Koda-Kimble 2004 Applied Therapeutics: The Clinical Use of Drugs, Lippincott Williams & Wilkins, ISBN 0781748453; Winter 2003 Basic Clinical Pharmacokinetics, 4^{th} ed., Lippincott Williams & Wilkins, ISBN 0781741475; Sharqel 2004 Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect can be achieved. If desired, the effective daily dose may be divided into multiple doses for purposes of administration. Consequently, single dose compositions may contain such amounts or submultiples thereof to make up the daily dose. It will be understood, however, that the total daily usage of the compounds or compositions of the present disclosure will be decided by an attending physician within the scope of sound medical judgment.

Again, each of the states, diseases, disorders, or conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an iron carbohydrate complex can occur as a single event or over a time course of treatment. For example, an iron carbohydrate complex can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for FM or FMS. For example, an iron carbohydrate complex can be administered prior to, concurrent with, or after administration of Lyrica® (Pregablin), Cymbalta® (duloxetine), or Savella® (milnacipran).

An iron carbohydrate complex can be administered simultaneously or sequentially with another agent, such as an antibiotic, an anti-inflammatory, or another agent. For example, an iron carbohydrate complex can be administered simultaneously with another agent, such as an antibiotic or an anti-inflammatory. Simultaneous administration can occur through administration of separate compositions, each containing one or more of an iron carbohydrate complex, an antibiotic, an anti-inflammatory, or another agent. Simultaneous administration can occur through administration of one composition containing two or more of a an iron carbohydrate complex, an antibiotic, an anti-inflammatory, or another agent. An iron carbohydrate complex can be administered sequentially with an antibiotic, an anti-inflammatory, or another agent. For example, an iron carbohydrate complex can be administered before or after administration of an antibiotic, an anti-inflammatory, or another agent.

Formulation

The agents or compositions described herein can be formulated by any conventional manner using one or more pharmaceutically acceptable carriers or excipients as described in, for example, Remington's Pharmaceutical Sciences 2005 (A. R. Gennaro, Ed.), 21st edition, ISBN: 0781746736, incorporated herein by reference in its entirety. Such formulations will contain a therapeutically effective amount of a biologically active agent described herein, which can be in purified form, together with a suitable amount of carrier so as to provide the form for proper administration to the subject.

In many cases, a single unit dose of iron carbohydrate complex may be delivered as a simple composition comprising the iron complex and the buffer in which it is dissolved. However, other products may be added, if desired, for example, to maximize iron delivery, preservation, or to optimize a particular method of delivery.

A “pharmaceutically acceptable carrier” can include any and all solvents, dispersion media, coatings, antibacterial or anti-fungal agents, isotonic or absorption delaying agents, or the like, compatible with pharmaceutical administration (see e.g., Banker, Modern Pharmaceutics, Drugs and the Pharmaceutical Sciences, 4th ed. (2002) ISBN 0824706749; Remington The Science and Practice of Pharmacy, 21st ed. (2005) ISBN 0781746736). Examples of such carriers or diluents include, but are not limited to, water, saline, Finger's solutions or dextrose solution. Supplementary active compounds can also be incorporated into the compositions. For intravenous administration, the iron carbohydrate complex can be diluted in normal saline to about 2 mg/ml to about 5 mg/ml. The volume of the pharmaceutical solution can be based on the safe volume for an individual subject, as determined by a medical professional.

An iron carbohydrate complex for administration can be formulated to be compatible with the intended route of administration, such as intravenous injection. Solutions or suspensions used for parenteral, intradermal or subcutaneous application can include a sterile diluent, such as water for injection, saline solution, polyethylene glycols, glycerine, propylene glycol or other synthetic solvents; antibacterial agents such as benzyl alcohol or methyl parabens; antioxidants such as ascorbic acid or sodium bisulfite; buffers such as acetates, citrates or phosphates, or agents for the adjustment of tonicity such as sodium chloride or dextrose. The pH can be adjusted with acids or bases, such as hydrochloric acid or sodium hydroxide. Preparations can be enclosed in ampules, disposable syringes or multiple dose vials made of glass or plastic.

Pharmaceutical compositions suitable for injection include sterile aqueous solutions or dispersions for the extemporaneous preparation of sterile injectable solutions or dispersion. For intravenous administration, suitable carriers include physiological saline, bacteriostatic water, Cremophor EL™ (BASF; Parsippany, N.J.) or phosphate buffered saline (PBS). The composition can be sterile or fluid so as to be administered using a syringe. Such compositions can be stable during manufacture or storage or can be preserved against contamination from microorganisms, such as bacteria or fungi. The carrier can be a dispersion medium containing, for example, water, polyol (such as glycerol, propylene glycol, or liquid polyethylene glycol), or other compatible, suitable mixtures. Various antibacterial or anti-fungal agents, for example, parabens, chlorobutanol, phenol, ascorbic acid, or thimerosal, can contain microorganism contamination. Isotonic agents such as sugars, polyalcohols, such as manitol, sorbitol, or sodium chloride can be included in the composition. Compositions that can delay absorption include agents such as aluminum monostearate or gelatin.

Sterile injectable solutions can be prepared by incorporating an iron carbohydrate complex in a suitable amount in an appropriate solvent with a single or combination of ingredients as required, followed by sterilization. Methods of preparation of sterile solids for the preparation of sterile injectable solutions include vacuum drying or freeze-drying to yield a solid containing the iron carbohydrate complex or any other desired ingredient.

Active compounds may be prepared with carriers that protect the compound against rapid elimination from the body, such as a controlled release formulation, including implants or microencapsulated delivery systems. Biodegradable or biocompatible polymers can be used, such as ethylene vinyl acetate, polyanhydrides, polyglycolic acid, collagen, polyorthoesters, or polylactic acid. Such materials can be obtained commercially from ALZA Corporation (Mountain View, Calif.) or NOVA Pharmaceuticals, Inc. (Lake Elsinore, Calif.), or prepared by one of skill in the art.

A dose of iron carbohydrate complex (as described herein) can be intravenously administered in a volume of pharmaceutically acceptable carrier of about 200 ml to about 300 ml of diluent. For example, a dose of iron carbohydrate complex can be intravenously administered in a volume of pharmaceutically acceptable carrier of about 1000 mg of elemental iron in about 250 ml of diluent. As another example, a single unit dose of iron carbohydrate complex may be intravenously administered in a volume of pharmaceutically acceptable carrier of about 1000 mg of elemental iron in about 215 ml of diluent.

A pharmaceutical composition for use in the methods described herein can contain an iron carboxymaltose (e.g., VIT-45) as the active pharmaceutical ingredient (API) with about 28% weight to weight (m/m) of iron, equivalent to about 53% m/m iron (III)-hydroxide, about 37% m/m of ligand, ≤6%, m/m of NaCl, and ≤10%, m/m of water.

Agents or compositions described herein can also be used in combination with other therapeutic modalities, as described further below. An iron carbohydrate complex may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces. Thus, in addition to the therapies described herein, one may also provide to the subject other therapies known to be efficacious for treatment of FM or FMS.

Administration

Agents or compositions described herein can be administered in a variety of methods well known in the arts. Administration can include methods disclosed in U.S. Pat. No. 7,754,702; U.S. Pat. No. 8,431,549; or U.S. Pat. No. 8,895,612, each incorporated herein by reference).

Methods of treatment of FM or FMS with an iron carbohydrate complex can include administration of the complex (e.g., in a single unit dosage or multiple combined dosages) of at least 200 mg (e.g., 300 mg, 400 mg, 500 mg, 600 mg, 700 mg, 800 mg, 900 mg, 1000 mg, or more) of elemental iron. For example, administration of an iron carbohydrate complex can provide up to about 2.5 grams of elemental iron, or more. Administration of single unit dosages can be, for example, over pre-determined time intervals or in response to the appearance or reappearance of symptoms. For example, the iron carbohydrate complex can be re-administered upon recurrence of at least one symptom of FM or FMS. As another example, the iron carbohydrate complex can be re-administered at some time period after the initial administration (e.g., after 4 days to 12 months).

Any route of delivery of an iron carbohydrate complex can be acceptable so long as iron from the iron complex can be released such that symptoms are treated. Administration can be parenteral, pulmonary, oral, topical, intradermal, intramuscular, intraperitoneal, intravenous, subcutaneous, intranasal, epidural, ophthalmic, buccal, or rectal administration.

A dose of iron carbohydrate complex can be administered parenterally, for example intravenously or intramuscularly. Intravenous administration can be delivered as a bolus or as an infusion. For example, the single unit dose of iron carbohydrate complex can be intravenously infused at a concentration of about 1000 mg elemental iron in about 200 ml to about 300 ml of diluent, preferably about 215 ml of diluent or about 250 ml of diluent. The iron carbohydrate complex can be intravenously injected as a bolus. For example, the iron carbohydrate complex can be intravenously injected as a bolus at a concentration of about 1000 mg elemental iron in about 200 ml to about 300 ml of diluent, preferably about 215 ml of diluent or about 250 ml of diluent. The iron carbohydrate complex can be intramuscularly infused at a concentration of, for example, about 1000 mg elemental iron in about 200 ml to about 300 ml of diluent, preferably, about 250 ml of diluent or about 215 ml of diluent. If applied as an infusion, the iron carbohydrate complex can be diluted with sterile saline (e.g., polynuclear iron (III)-hydroxide 4(R)-(poly-(1→4)-O-α-glucopyranosyl)-oxy-2(R),3(S),5(R),6-tetrahydroxy-hexanoate (“VIT-45”) 0.9% m/V NaCl or 500 mg iron in up to 250 mL NaCl). The iron carbohydrate complex can be intravenously injected as a bolus without dilution. As an example, the iron carbohydrate complex can be intramuscularly injected at a concentration of about 500 mg elemental iron in less than about 10 ml diluent, preferably about 5 ml.

One skilled in the art can tailor the total iron dose required for a subject while avoiding iron overload, so as to avoid overdosing.

An iron carbohydrate complex can be delivered as a single unit dosage or a series of single unit dosages. For treatment methods disclosed herein, an iron carbohydrate complex can have a dosage level of at least 0.1 grams, at least 0.2 grams, at least 0.3 grams, at least 0.4 grams, at least 0.5 grams, 0.6 grams, at least 0.7 grams; at least 0.8 grams; at least 0.9 grams; at least 1.0 grams; at least 1.1 grams; at least 1.2 grams; at least 1.3 grams; at least 1.4 grams; at least 1.5 grams; at least 1.6 grams; at least 1.7 grams; at least 1.8 grams; at least 1.9 grams; at least 2.0 grams; at least 2.1 grams; at least 2.2 grams; at least 2.3 grams; at least 2.4 grams; or at least 2.5 grams of elemental iron, or more. For example, a dosage can be at least 1.0 grams of elemental iron. As another example, a dosage can be at least 1.5 grams of elemental iron. As a further example, a dosage can be at least 2.0 grams of elemental iron. In yet another example, a dosage can be at least 2.5 grams of elemental iron.

For example, an iron carbohydrate complex can have a dosage level of at least about 0.1 grams, at least about 0.2 grams, at least about 0.3 grams, at least about 0.4 grams, at least about 0.5 grams, 0.6 grams, at least about 0.7 grams; at least about 0.8 grams; at least about 0.9 grams; at least about 1.0 grams; at least about 1.1 grams; at least about 1.2 grams; at least about 1.3 grams; at least about 1.4 grams; at least about 1.5 grams; at least about 1.6 grams; at least about 1.7 grams; at least about 1.8 grams; at least about 1.9 grams; at least about 2.0 grams; at least about 2.1 grams; at least about 2.2 grams; at least about 2.3 grams; at least about 2.4 grams; or at least about 2.5 grams of elemental iron.

As another example, an iron carbohydrate complex can have a dosage level of about 0.1 grams, about 0.2 grams, about 0.3 grams, about 0.4 grams, about 0.5 grams, 0.6 grams, about 0.7 grams; about 0.8 grams; about 0.9 grams; about 1.0 grams; about 1.1 grams; about 1.2 grams; about 1.3 grams; about 1.4 grams; about 1.5 grams; about 1.6 grams; about 1.7 grams; about 1.8 grams; about 1.9 grams; about 2.0 grams; about 2.1 grams; about 2.2 grams; about 2.3 grams; about 2.4 grams; or about 2.5 grams of elemental iron.

As another example, an iron carbohydrate complex can have a dosage level of up to about 0.1 grams, up to about 0.2 grams, up to about 0.3 grams, up to about 0.4 grams, up to about 0.5 grams, 0.6 grams, up to about 0.7 grams; up to about 0.8 grams; up to about 0.9 grams; up to about 1.0 grams; up to about 1.1 grams; up to about 1.2 grams; up to about 1.3 grams; up to about 1.4 grams; up to about 1.5 grams; up to about 1.6 grams; up to about 1.7 grams; up to about 1.8 grams; up to about 1.9 grams; up to about 2.0 grams; up to about 2.1 grams; up to about 2.2 grams; up to about 2.3 grams; up to about 2.4 grams; or up to about 2.5 grams of elemental iron.

An appropriate dosage level can also be determined on the basis of patient weight. For example, an appropriate dosage level can be at least 9 mg of elemental iron per kg body weight, at least 10.5 mg/kg, at least 12 mg/kg, at least 13.5 mg/kg, at least 15 mg/kg, at least 16.5 mg/kg, at least 18 mg/kg, at least 19.5 mg/kg, at least 21 mg/kg, at least 22.5 mg/kg, at least 24 mg/kg, at least 25.5 mg/kg, at least 27 mg/kg, at least 28.5 mg/kg, at least 30 mg/kg, at least 31.5 mg/kg, at least 33 mg/kg, at least 34.5 mg/kg, at least 36 mg/kg, or at least 37.5 mg/kg.

In some embodiments, a dosage can be administered in 15 minutes or less. For example, a dosage can be administered in about 14 minutes or less, about 13 minutes or less, about 12 minutes or less, about 11 minutes or less, about 10 minutes or less, about 9 minutes or less, about 8 minutes or less, about 7 minutes or less, about 6 minutes or less, about 5 minutes or less, about 4 minutes or less, about 3 minutes or less, or about 2 minutes or less.

Administration of an iron carbohydrate complex can occur as a one-time delivery of a single unit dose or over a course of treatment involving delivery of multiple single unit doses. Multiple single unit doses can be administered, for example, over pre-determined time intervals or in response to the appearance or reappearance of symptoms. The frequency of dosing depends on the disease or disorder being treated, the response of each individual patient, or the administered amount of elemental iron. An appropriate regime of dosing adequate to allow the body to absorb the iron from the bloodstream can be, for example, a course of therapy once every day to once every eighteen months.

Such consecutive single unit dosing can be designed to deliver a relatively high total dosage of iron over a relatively low period of time. For example, a single unit dose (e.g., 1000 mg) can be administered every 24 hours. As illustration, a total dose of 2000, 2500, 3000, 3500, 4000, 4500, or 5000 mg of elemental iron can be delivered via consecutive daily single unit doses of up to about 1000 mg of elemental iron. Given that a relatively high single unit dose (e.g., 500 mg, or 1000 mg) can be intravenously introduced into a subject in a concentrated form over, for example, a few minutes, such administrative protocol provides a practitioner or subject with an effective, efficient, or safe means to deliver elemental iron.

As another example, a single unit dose can be administered every 3-4 days. As a further example, a single unit dose can be administered once per week. Alternatively, the single unit doses of an iron carbohydrate complex may be administered ad hoc, that is, as symptoms reappear, as long as safety precautions are regarded as practiced by medical professionals.

It will be understood, however, that the specific dose or frequency of administration for any particular subject may be varied and depends upon a variety of factors, including the activity of the employed iron carbohydrate complex, the metabolic stability or length of action of that complex, the age, body weight, general health, sex, diet, mode or time of administration, rate of excretion, drug combination, the severity or nature of the particular condition, or the host undergoing therapy.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to an iron carbohydrate complex. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, or envelopes that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, or the like.

In certain embodiments, kits can be supplied with instructional materials. Instructions may be printed on paper or other substrate, or may be supplied as an electronic-readable medium, such as a floppy disc, mini-CD-ROM, CD-ROM, DVD-ROM, Zip disc, videotape, audio tape, or the like. Detailed instructions may not be physically associated with the kit; instead, a user may be directed to an Internet web site specified by the manufacturer or distributor of the kit.

Compositions or methods described herein utilizing molecular biology protocols can be according to a variety of standard techniques known to the art (see, e.g., Sambrook and Russel 2006 Condensed Protocols from Molecular Cloning: A Laboratory Manual, Cold Spring Harbor Laboratory Press, ISBN-10: 0879697717; Ausubel 2002 Short Protocols in Molecular Biology, 5th ed., Current Protocols, ISBN-10: 0471250929; Sambrook and Russel 2001 Molecular Cloning: A Laboratory Manual, 3d ed., Cold Spring Harbor Laboratory Press, ISBN-10: 0879695773; Elhai and Wolk 1988 Methods in Enzymology 167, 747-754; Studier 2005 Protein Expr Purif. 41(1), 207-234; Gellissen 2005 Production of Recombinant Proteins: Novel Microbial and Eukaryotic Expression Systems, Wiley-VCH, ISBN-10: 3527310363; Baneyx 2004 Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Efficacy and Safety of Iron Carboxymaltose (Injectafer®) in the Treatment of Iron Deficient Patients with Fibromyalgia

The following example describes the outline for a blinded, randomized, placebo-controlled study investigating the efficacy and safety of iron (ferric) carboxymaltose in the treatment of iron deficient patients with Fibromyalgia (FM). The primary objective of this study was to evaluate the efficacy and safety of an intravenously administered iron carboxymaltose in subjects with Fibromyalgia.

Study Design

This was a blinded, randomized, placebo-controlled study. All subjects who met the inclusion requirements and no exclusion criteria were entered into an up to 14 day Screening Phase to obtain 80 eligible subjects for study drug treatment. Eligible subjects were randomized in a 1:1 ratio to receive iron carboxymaltose or placebo on Days 0 and 5. All treated subjects were followed for efficacy and safety for 42 days. Subjects visited the clinic on Days 0 and 5 for treatment, and then on Days 14, 28, and 42. The subject's participation in the study was for approximately 42 Days from Day 0.

Study Drug Treatment

The duration of treatment phase is 5 days. On Day 0 (start of Treatment Phase) Group A received a 15 mg/kg (up to 750 mg) undiluted blinded dose of IV Iron carboxymaltose at 100 mg/minute. Group B received a blinded placebo (15 cc of Normal Saline [NS]) IV push at 2 ml/minute. On Day 5 Group A received a 15 mg/kg (up to 750 mg) undiluted blinded dose of IV iron carboxymaltose at 100 mg/minute. Group B received a blinded placebo (15 cc of Normal Saline [NS]) IV push at 2 ml/minute.

Efficacy and Safety Follow-Up

The duration of the study was about 42 days. After treatment on Days 0 and 5, subjects visited the clinic on Days 14, 28, and 42.

Efficacy and Safety Evaluations

Efficacy evaluations included (see e.g., TABLE 1, Schedule of Events, for details):

1. Revised Fibromyalgia Impact Questionnaire (FIQR)

2. Brief Pain Inventory (short form)

3. Medical Outcome Study (MOS) Sleep Scale

4. Fatigue Visual Numeric Scale

5. Change from Baseline for iron indices (percentage serum transferrin saturation (TSAT), Ferritin, serum iron)

Safety Evaluations include (see e.g., TABLE 1, Schedule of Events, for details):

1. Adverse events

2. Laboratory assessments, including Hematology (hemoglobin (Hgb), hematocrit (Hct), red blood cell count (RBC), white blood cell count (WBC), mean corpuscular volume (MCV), mean corpuscular hemoglobin (MCH), mean corpuscular hemoglobin concentration (MCHC), red cell distribution width (RDW), platelets, differential count), Iron indices (serum iron, serum ferritin, and total iron binding capacity (TIBC), transferrin saturation (TSAT)), Clinical chemistry (sodium, potassium, chloride, blood urea nitrogen (BUN), creatinine, albumin, alkaline phosphatase, total bilirubin, gamma glutaminyl transferase (GGT), aspartate aminotransferase (AST), alanine aminotransferase (ALT), lactic dehydrogenase (LDH), calcium, phosphorus, glucose, bicarbonate) and other (hepcidin)

3. Vital signs

4. Physical examinations

Intervention

Intervention was defined, in this example, as the initiation of a new treatment or increase of a previously prescribed treatment by a physician used to specifically relieve the symptoms of fibromyalgia following the subject's assessment that the fibromyalgia symptoms were intolerable. Any subject who had an intervention was no longer to be evaluated for efficacy starting at the time of the intervention, however these subjects remained in the study and continued to be evaluated for safety.

Inclusion Criteria

1. Male or female subjects ≥18 years of age, able to give informed consent to the study

2. Fibromyalgia diagnosis based on the 2011 modification of the American College of Rheumatology (ACR) 2010 preliminary criteria for diagnosing fibromyalgia (2011ModCr)

3. A baseline score ≥60 on the FIQR

4. Subject must have been on a stable dose of current medications to treat fibromyalgia, including pain medicines, antidepressants, sleep medications for at least 1 month

5. Subject must have been on a stable dose of current narcotic medication for at least 30 days prior to randomization

6. Subjects at risk for pregnancy must have had a negative pregnancy test at baseline and been practicing an acceptable form of birth control, had a hysterectomy or tubal ligation, or otherwise been incapable of pregnancy, or practiced any of the following methods of contraception for at least one month prior to study entry: hormonal contraceptives, spermicide and barrier, intrauterine device, or partner sterility

Exclusion Criteria

1. Parenteral iron used within 4 weeks prior to screening

2. History of >10 blood transfusions in the past 2 years

3. Anticipated need for blood transfusion during the study

4. Known hypersensitivity reaction to any component of iron carboxymaltose.

5. Current or acute or chronic infection other than viral upper respiratory tract infection

6. Malignancy (other than basal or squamous cell skin cancer or the subject has been cancer free for ≥5 years)

7. Active inflammatory arthritis (e.g., rheumatoid arthritis, systemic lupus erythematosus (SLE))

8. Pregnant or lactating women

9. Severe peripheral vascular disease with significant skin changes

10. Seizure disorder currently being treated with medication

11. Baseline ferritin ≥50 ng/mL

12. Baseline TSAT ≥20%

13. History of hemochromatosis or hemosiderosis or other iron storage disorders

14. Known positive hepatitis with evidence of active disease

15. Hemoglobin greater than the upper limit of normal

16. Calcium or phosphorous outside the normal range

17. Known positive hepatitis B antigen (HBsAg) or hepatitis C viral antibody (HCV) with evidence of active hepatitis (i.e., AST/ALT greater than the upper limit of normal)

18. Known positive HIV-1/HIV-2 antibodies (anti-HIV)

19. Received an investigational drug within 30 days before randomization

20. Chronic alcohol or drug abuse within the past 6 months

21. Any other pre-existing laboratory abnormality, medical condition or disease which, in view of the investigator participation in this study, may put the subject at risk

22. Subject unable to comply with the study requirements

Study Endpoints

The primary efficacy variable is the proportion of patients with a ≥13 point improvement in FIQR score on Day 42.

Other Efficacy Endpoints:

1. FIQR scores change from baseline at each time point and to lowest

2. Brief Pain Inventory total score change from baseline at each time point

3. MOS Sleep scale total score change from baseline at each time point

4. Fatigue Visual Numeric Scale change from baseline at each time point

5. Proportion of subjects required intervention for fibromyalgia and time to fibromyalgia intervention

6. Proportion of subjects with relapse (intervention)

7. Change from Baseline for iron indices (TSAT, Ferritin, serum iron) to each time point

The safety endpoints include:

1. Incidence of treatment emergent adverse events, incidence of serious adverse events, and incidence of severe adverse events

2. Change in clinical laboratory tests

3. Change in vital signs

Blinding

All subjects, investigators, and study personnel were blinded to the content of study drug with the exception of the un-blinded study personnel who was responsible for the following:
Randomizing the subject on Day 0
Preparing, concealing, and administering the study drug on Day 0 and 5 (as iron carboxymaltose is reddish-brown in color and slightly viscous)
Completing the Study Drug Accountability Form, study drug dosing record, and applicable case report form pages

Study Duration
Screening Phase: up to 14 days
Treatment Phase: 5 days
Follow-up Phase: approximately 37 days

Number of Subjects and Study Sites Approximately 80 subjects (40 per group) were enrolled at one study site in the United States.

Sample Size

If the proportion of responders (≥13 point improvement in FIQR score on Day 42) is 30% in the placebo group, a sample size of 40 patients per group provided 78% power to detect a 2.0-fold increase in the responder rate (i.e., 30% versus 60%) and >95% power to detect a 2.5-fold increase in the responder rate (i.e., 30% versus 75%).

Statistical Methods

Treatment group differences for proportions were assessed with the chi-square test. Treatment group differences for means were assessed with the analysis of covariance with treatment as a fixed factor and with baseline score as a covariate.

For the proportion of responders, subjects who discontinued or completed the study were considered as non-responders. For comparison of means, the primary imputation method was last observation carried forward. Sensitivity analyses assessed the impact of missing values on inferences.

Analyses of safety data were descriptive and no formal statistical comparisons were made.

### Example 2: Iron Carboxymaltose (e.g., Injectafer®) in Humans

The following example shows a clinical pharmacokinetic study (VIT-IV-CL-001) using positron emission tomography (PET) demonstrated a fast initial elimination of radioactively labeled iron (Fe)⁵²Fe/⁵⁹Fe iron carboxymaltose (e.g., Injectafer®) from the blood, with rapid transfer to the bone marrow and rapid deposition in the liver and spleen.

Eight hours after administration, 5% to 20% of the injected amount was still in the blood, compared with 2% to 13% for iron sucrose. The projected terminal half-life (t½) was calculated to approximately 16 hours, compared to 3 to 4 days for iron dextran and 6 hours for iron sucrose. An ascending dose pharmacokinetic study (VIT-IV-CL-002), demonstrated that following the 500 mg and 1,000 mg iron carboxymaltose dose, the majority of the iron carboxymaltose complex was utilized or excreted by 72 hours.

### Example 3: Study Design and Rationale

The primary objective of this study is to evaluate the efficacy and safety of an IV Iron, Injectafer® in subjects with Fibromyalgia.

Trial Design

This was a blinded, randomized, placebo-controlled study. All subjects who meet the inclusion requirements and no exclusion criteria were entered into an up to 14 day Screening Phase to get 40 eligible subjects for study drug treatment. Eligible subjects were randomized in a 1:1 ratio to receive iron carboxymaltose (Injectafer®) or placebo (normal saline, obtained by the investigator through commercial sources and the lot numbers were not collected) on Days 0 and 5. All treated subjects were followed for efficacy and safety for 42 days. Subjects visited the clinic on Days 0 and 5 for treatment, and then on Days 14, 28, and 42. The subject's participation in the study was for approximately 42 Days from Day 0. All Injectafer® vials were kept by the study staff and returned to the supplier after drug accountability had been completed by the monitor.

The duration of treatment phase was 5 days. On Day 0 (start of Treatment Phase) Group A received a 15 mg/kg (up to 750 mg) undiluted blinded dose of IV Injectafer® at 100 mg/minute. The total dose of Injectafer® was consistent with FDA-approved instructions for dosage and administration. Group B received a blinded placebo (15 cc of Normal Saline [NS]) IV push at 2 ml/minute. On Day 5 Group A received a 15 mg/kg (up to 750 mg) undiluted blinded dose of IV Injectafer® at 100 mg/minute. Group B received a blinded placebo (15 cc of Normal Saline [NS]) IV push at 2 ml/minute. Starting and stopping times of study drug administration and the total dose and/or volume administered were documented.

The duration of the study was about 42 days. After treatment on Days 0 and 5 subjects visited the clinic on Days 14, 28, and 42.

Rationale Rationale for trial design: Injectafer® is a non-dextran IV iron recently approved by the United States Food and Drug Administration (FDA). An increased prevalence of iron deficiency has been seen in patients with fibromyalgia. It was hypothesized that correcting this iron deficiency may improve or alleviate some fibromyalgia symptoms.

Blinding: all subjects, investigators, and study personnel were blinded to the content of study drug with the exception of the un-blinded study personnel who were responsible for the following:
Randomizing the subject on Day 0
Preparing, concealing, and administering the study drug on Day 0 and 5 (as Injectafer® is reddish-brown in color and slightly viscous).
Completing the Study Drug Accountability Form, study drug dosing record, and applicable case report form pages.

TABLE 1
Schedule of Events: Screening Treatment
Efficacy and Safety Follow-up

| | | | | Efficacy and Safety | | |
|---|---|---|---|---|---|---|
| | Screening | Treatment | | Follow-up | | |
| Study Day | −14 to −4 | 0 | 5 | 14 | 28 | 42 |
| Informed Consent | X | | | | | |
| Eligibility | X | X | | | | |
| Medical History | X | | | | | |
| D/C oral iron products | X | | | | | |
| Physical exam | X | | | | | X |
| Vital Signs | X | X³ | X² | | | X¹ |
| Weight without shoes | X | | | | | |
| FIQR Scale | | X | | X | X | X |
| Brief Pain Inventory | | X | | X | X | X |
| MOS Sleep Scale | | X | | X | X | X |
| Fatigue Visual | | X | | X | X | X |
| Numeric Scale | | | | | | |
| IRLS Scale | X | | | | | |
| Hematology | X | | | | | X |
| Iron Indices | X | | | | | X |
| Chemistry | X | | | | | X |
| Pregnancy test⁴ | X | | | | | X |
| Hepcidin | X | | | | | X |
| Study Drug Dosing | | X | X | | | |
| Concomitant Meds | X | X | X | X | X | |
| Adverse Events⁵ | | X | X | X | X | X |

¹Vital Signs includes sitting blood pressure (BP) and heart rate
²On study drug dosing days sitting vital signs including blood pressure and heart rate were collected pre dosing, immediately and 30 minutes post dosing. Body temperature was also collected pre dosing on Days 0 and 5.
³If the subjects phosphorous was below the lower limit of normal (LLN) at Day 42 the subject should have returned for repeat phosphorous until the value is back within normal limits (WNL's).
⁴For women of child bearing potential (negative results must have be obtained prior to randomization)
⁵Adverse event assessments started at the time of the first dose of study drug. All events noted prior to the 1st dose of study drug should have been considered history and captured on the medical history page of the case report form (CRF).

### Example 4: Subject Selection

The following example describes the subject selection process and criteria for subject selection.

Number and Type of Subjects

Approximately 80 subjects who gave written informed consent with a diagnosis of Fibromyalgia who fulfilled the inclusion criteria, did not meet any of the exclusion criteria were randomized into Group A (Injectafer®) or Group B (normal saline).

If the proportion of responders (≥13 point improvement in FIQR score on Day 42) is 30% in the placebo group, a sample size of 40 patients per group provided 78% power to detect a 2.0-fold increase in the responder rate (i.e., 30% versus 60%) and >95% power to detect a 2.5-fold increase in the responder rate (i.e., 30% versus 75%).

Screening Phase

Once a subject entered the screening phase, they were assigned a unique screening number. From the time of consent until the start of treatment which includes IV Injectafer® or placebo the subject did not receive any form of iron outside of the study (intravenous iron from 30 days prior to consent or oral iron including multivitamins with iron from time of consent).

Entry Criteria

Inclusion Criteria:

1. Male or female subject's ≥18 years of age, able to give informed consent to the study.

2. Fibromyalgia diagnosis based on the 2011 modification of the American College of Rheumatology (ACR) 2010 preliminary criteria for diagnosing fibromyalgia (2011ModCr)

3. A baseline score ≥60 on the FIQR

4. Subject must have been on a stable dose of current medications to treat fibromyalgia, including pain medicines, antidepressants, and sleep medications for at least 1 month.

5. Subject must have been on a stable dose of current narcotic medication for at least 30 days prior to randomization

6. Subjects at risk for pregnancy must have had a negative pregnancy test at baseline and been practicing an acceptable form of birth control, have had a hysterectomy or tubal ligation, or otherwise been incapable of pregnancy, or have practiced any of the following methods of contraception for at least one month prior to study entry: hormonal contraceptives, spermicide and barrier, intrauterine device, or partner sterility.

Exclusion Criteria:

1. Parenteral iron use within 4 weeks prior to screening

2. History of >10 blood transfusions in the past 2 years

3. Anticipated need for blood transfusion during the study

4. Known hypersensitivity reaction to any component of Injectafer® (iron carboxymaltose)

5. Current or acute or chronic infection other than viral upper respiratory tract infection

6. Malignancy (other than basal or squamous cell skin cancer or the subject has been cancer free for ≥5 years)

7. Active inflammatory arthritis (e.g. rheumatoid arthritis, SLE)

8. Pregnant or lactating women

9. Severe peripheral vascular disease with significant skin changes

10. Seizure disorder currently being treated with medication

11. Baseline ferritin ≥50 ng/mL

12. Baseline TSAT ≥20%

13. History of hemochromatosis or hemosiderosis or other iron storage disorders

14. Known positive hepatitis with evidence of active disease

15. Hemoglobin greater than the upper limit of normal

16. Calcium or phosphorous outside the normal range

17. Known positive hepatitis B antigen (HBsAg) or hepatitis C viral antibody (HCV) with evidence of active hepatitis (i.e., AST/ALT greater than the upper limit of normal)

18. Known positive HIV-1/HIV-2 antibodies (anti-HIV)

19. Received an investigational drug within 30 days before randomization

20. Chronic alcohol or drug abuse within the past 6 months

21. Any other pre-existing laboratory abnormality, medical condition or disease which in view of the investigator participation in this study may put the subject at risk

22. Subject unable to comply with the study requirements

Subject Assignment and Randomization Process

Subjects that met all the inclusion requirements, and no exclusionary criteria, were offered participation in the study. Subjects were randomized in a 1:1 ratio to either Group A (Injectafer®) or Group B (normal saline).

Group A: Subjects received a 15 mg/kg (up to 750 mg) undiluted blinded dose of IV Injectafer® at 100 mg/minute on study Days 0 and 5.

Group B: Subjects received a blinded placebo (15 cc of Normal Saline [NS]) IV push at 2 ml/minute on study Days 0 and 5.

Withdrawal from Study

Any subject who wished to withdraw from the study may have done so at any time without the need to justify their decision. The investigator may have withdrawn a subject from the trial at any time if it was felt to be in the best interest of the subject.

At the times of withdrawal, procedure for the Day 42 visit were to have been performed regardless of whether the subject had completed the study drug treatment. In the event the subject had received any study drug; the subject should have been contacted on Day 42 for follow-up to assess adverse events, if possible.

Intervention

Intervention was defined as the initiation of a new treatment or increase of a previously prescribed treatment by a physician used to specifically relieve the symptoms of fibromyalgia following the subject's assessment that the fibromyalgia symptoms were intolerable. Any subject who had an intervention was no longer evaluated for efficacy starting at the time of the intervention, however these subjects remained in the study and continued to be evaluated for safety.

### Example 5: Study Drug

The following example describes the formulation, packaging, storage, administration, precautions, and accountability of the drug.

Formulation Packaging and Storage

All medication to be used in this study was prepared according to Good Manufacturing Practices (GMP).

Injectafer® (Ferric Carboxymaltose injection) was supplied as 15 mL vials, containing 750 mg of iron as 5% w/v (weight/volume) iron containing a polynuclear iron(III)-hydroxide 4(R)-(poly-(1→4)-O α-D-glucopyranosyl)-oxy-2 (R), 3(S), 5(R), 6-tetrahydroxy-hexonate complex in a solution of water for injection (50 mg/mL) and was labeled according to FDA investigational regulatory requirements.

All study drugs were to be kept in a secure place at the investigational site, and stored at room temperature (see: USP). Injectafer® was not to be frozen. Vials were not used for more than 1 dose or for more than 1 subject.

Drug Administration/Regimen

The Principal Investigator or designee supervised administration of the study drug to subjects.

Group A: Subjects received a 15 mg/kg (up to 750 mg) undiluted blinded dose of IV Injectafer® at 100 mg/minute on study Days 0 and 5.

Group B: Subjects received a blinded placebo (15 cc of Normal Saline [NS]) IV push at 2 ml/minute on study Days 0 and 5.

IV Iron Precautions

When administering IV Iron, the following precautions were taken:
The subject was clinically evaluated prior to drug administration to assess the development of clinically significant conditions.
The vials were visually inspected for particulate matter and discoloration before each use; if noted, the vial was not used and the Investigator or his/her designee notified the sponsor, or sponsor's designee, for replacement of the study drug and for directions to return the unused vial.
Sitting heart rate and blood pressure were assessed pre-, immediately post, and 30 minutes post administration. If the subject was an outpatient, they were discharged from the site by the Investigator only if there were no significant signs or symptoms 30 minutes after administration was complete.
Serious hypersensitivity reactions, including anaphylactic-type reactions, some of which have been life-threatening and fatal, have been reported in patients receiving IV Iron therapies. Subjects may present with shock, clinically significant hypotension, loss of consciousness, and/or collapse. If hypersensitivity reactions or signs of intolerance occurred during administration, IV iron administration was to be stopped immediately. The subject was to be monitored for at least 30 minutes, and until clinically stable following completion of infusion. IV iron was only administered when personnel and therapies were immediately available for treatment of serious hypersensitivity reactions. Most reactions associated with intravenous iron preparations occur within 30 minutes of the completion of the iron infusion.

Drug Accountability

The investigator kept adequate records of the receipt, administration and return of Injectafer® and/or normal saline, and did not allow Injectafer® or normal saline to be used for purposes other than as directed by this protocol. The investigator agreed that he/she would not supply study medication to any persons other than those screened and randomized in the study, or to investigators not listed on the FDA 1572. When the study was completed, or if it was prematurely terminated, a final inventory of all clinical supplies must have been compiled and the remainder of used and unused Injectafer® and normal saline were returned. All data regarding Injectafer® and normal saline were to be recorded on the Drug Accountability Forms provided by the sponsor.

Investigators kept adequate records of the administration and disposition of IV Injectafer® and normal saline used for patients selected for the trial.

Concomitant Medication Concomitant medications along with the route of administrations and duration were recorded in the case report form (CRF). No additional iron preparations (IV iron from 30 days prior to consent or oral iron including multivitamins with iron, from time of consent), were allowed. No prophylactic medications were to be administered prior to Injectafer® administration without prior approval.

The subject must have been on a stable dose (at least one month) of current medications to treat fibromyalgia, including pain medications, antidepressants, and sleep medications. The subject must have been on a stable dose of current narcotic medications for 30 days prior to randomization.

### Example 6: Study Procedures

The following example describes the study procedures including obtaining consent, subject screening, subject treatment timeline, and lab analyses.

Informed Consent

Prior to any study specific procedures, the investigator explained to each subject the nature of the study, its purpose, procedures to be performed, expected duration, and the benefits and risks of study participation. After this explanation the subject must have voluntarily signed an informed consent statement. The subject was given a copy of the signed consent form.

Screening

Each subject who qualified for inclusion underwent the following clinical evaluations to confirm their eligibility for the study:
Assign a screening number
Medical history, including prior iron therapy use
Weight without shoes
Basic physical exam including assessment of skin, cardiovascular, pulmonary, abdominal and central nervous system
Vital signs (including sitting heart rate and blood pressure)
Serum pregnancy test for women of child bearing potential (negative results must have been obtained prior to randomization)
Hematology, iron indices, chemistry and hepcidin levels
Completion of international restless leg syndrome (IRLS) Scale

Subjects who did not meet the entry criteria were documented as a screen failure. A subject may have been re-screened, one time, once it was believed that they would qualify for the study entry. The subject needed to re-sign a new consent form and all screening procedures needed to be repeated.

Study Visit Day 0-Day 42

Day 0

The following occurred prior to randomizing the subject:
Re-verified inclusion and exclusion criteria
Updated any relevant history
Concomitant medication assessment

Once it was confirmed that the subject continued to meet the criteria, all eligible subjects were randomized to either Group A (Injectafer®) or Group B (Normal Saline) in a 1:1 ratio based on a predetermined randomization schedule via EDC system. After assignment of treatment the following occurred:
All subjects (prior to drug administration, if applicable) on Day 0
Vital signs: temperature, sitting blood pressure and heart rate will be obtained
Concomitant medications
Completion of FIQR Scale, Brief Pain Inventory, MOS Sleep Scale and Fatigue Visual Numeric Scale,

Group A:
Verified the amount of single Injectafer® dose
Documented start and stop time of Injectafer® administration and the total dose administered
Obtained sitting heart rate and blood pressure immediately post and 30 minutes post Injectafer® administered
Adverse event assessment (starting at the beginning of Injectafer® injection)

Group B:
Verified amount of normal saline dose
Documented start and stop time of normal saline injection and the total volume administered
Obtained sitting heart rate and blood pressure immediately post and 30 minutes post administration
Adverse event assessment (starting at the beginning of the injection) Day 5 Visit
The following occurred on Day 5 visits for all Subjects prior to dosing:
Vital Signs
Concomitant medication assessment
Adverse Events Assessment

Group A:
Verified amount of Injectafer® dose
Documented start and stop time of Injectafer® administration and the total dose administered
Obtained sitting heart rate and blood pressure immediately post and 30 minutes post Injectafer® administration

Group B:
Verified amount of normal saline dose
Documented start and stop time of administration and the total dose administered
Obtained sitting heart rate and blood pressure immediately post and 30 minutes post administration

Day 14, and 28 Visits

The following occurred on Days 14 and 28 for all Subjects:
Completion of FIQR Scale, Brief Pain Inventory, MOS Sleep Scale, Fatigue Visual Numeric Scale
Concomitant medication assessment
Adverse Events Assessment

Day 42 End of Study Visit

The following occurred on Day 42:
Vital signs
Completion of FIQR Scale, Brief Pain Inventory, MOS Sleep Scale, Fatigue Visual Numeric Scale
Hematology, Iron Indices, Chemistry, Serum pregnancy test and Hepcidin levels
Physical exam

Laboratory Assessment

Serum samples for laboratory analysis were obtained at all appropriate visits and were analyzed by the local laboratory. All serum laboratory testing was provided to the physician for review and assessment. If the Investigator wished to obtain a follow-up of an abnormal Day 42 laboratory, this laboratory was to be obtained after notification of the Sponsor. If a subject's phosphorous was below the LLN at Day 42 the subject should have returned (as directed by the Investigator) for repeat phosphorous until the value was back WNL's. The laboratory assessments were:
Hematology: Hb, Hct, RBC, WBC, MCV, MCH, MCHC, RDW, platelets, differential count, and reticulocyte count/reticulocyte index
Chemistry: Sodium, potassium, chloride, BUN, creatinine, albumin, alkaline phosphate, total bilirubin, GGT, AST, ALT, LDH, calcium, phosphorus, glucose, bicarbonate, and magnesium
Iron Indices: Serum iron, serum ferritin, and total iron binding capacity (TIBC), and percentage serum transferrin saturation (TSAT)
Other: Serum pregnancy test, Hepcidin, and CRP

### Example 7: Assessment of Safety

The following example describes the safety assessment of Injectafer® (iron carboxymaltose).

Adverse Events

Any untoward medical event experienced by a subject during the course of this clinical trial whether or not it was related to the investigational product, at any dose, must have been recorded on the Adverse Event page of the CRF.

For any laboratory abnormality that the physician judged to be a clinically significant worsening from the baseline value was considered an adverse event and was recorded on the Adverse Events page of the CRF. If the laboratory value was outside the normal range, but not an adverse event, the investigator was to comment on the findings in the source documentation.

To quantify the severity of adverse events, the National Cancer Institute (NCI) Common Terminology Criteria for Adverse Events (CTCAE), Version 4 was used to grade all events. These criteria are provided in the procedure manual.

If a CTCAE criterion does not exist, the investigator used TABLE 2 to assign the adverse event grade.

TABLE 2
Grading of Adverse Event Severity as per CTCAE v 4

| Grade | Adjective | Description |
|---|---|---|
| 1 | Mild | Asymptomatic or mild symptoms; clinical or |
| | | diagnostic observations only; intervention not |
| | | indicated. |
| 2 | Moderate | Moderate; minimal, local or noninvasive |
| | | intervention indicated; limiting age-appropriate |
| | | instrumental activities of daily living (i.e., |
| | | preparing meals, shopping for groceries or |
| | | clothes, using the telephone, managing money, |
| | | etc.) |
| 3 | Severe | Severe or medically significant but not |
| | | immediately life-threatening; hospitalization or |
| | | prolongation of hospitalization indicated; |
| | | disabling; limiting self care activities of daily |
| | | living (i.e., bathing, dressing and undressing, |
| | | feeding self, using the toilet, taking |
| | | medications, and not bedridden.) |
| 4 | Life-threating | Life-threatening consequences; urgent |
| | | intervention indicated. |
| 5 | Death | Results in Death due to the AE |

Timing: Non-serious adverse events were reported from the initial treatment with study drug through the completion of study Day 42. Adverse Events (AEs) were captured during the follow-up phone call at Day 42 for subjects who were randomized and early terminated from the trial. All ongoing adverse events related to study drug should have been followed until they were no longer related, have taken a confounding medication or return to baseline grade.

Relationship/Causality: The Investigator was asked to document his/her opinion of the relationship of the event to the study drug as follows:

NONE: There was no evidence of any causal relationship.

UNLIKELY: There was little evidence to suggest there was a causal relationship. There was another reasonable explanation for the event (e.g., the subject's clinical condition, other concomitant treatments).

POSSIBLE: There was some evidence to suggest a causal relationship (i.e., there was a reasonable possibility that the adverse experience may have been caused by the agent). However, the influence of other factors may have contributed to the event (e.g., the subject's clinical condition, other concomitant events).

PROBABLE: There was evidence to suggest a causal relationship, and the influence of other factors was unlikely.

Reporting of Adverse Events

Adverse experiences were elicited by nonspecific questions such as “Have you noticed any problems?” Subjects were encouraged to report adverse events at their onset. Any adverse experience spontaneously reported by, elicited from the subject, or observed by the physician or study staff was to be recorded on the appropriate Adverse Events page of the CRF. The investigator was to record the date and time of onset, severity, the relationship to study medication, the date and time of resolution (or the fact that the event was still continuing), the action taken, and the outcome of the adverse experience on the Adverse Events page of the CRF. Whenever possible, the investigator was to group together, into a single term, signs and symptoms which constituted a single diagnosis. For example, cough, rhinitis, and sneezing might have been grouped together as “upper respiratory infection.”

Serious Adverse Events

An adverse event was classified as SERIOUS if it met any one of the following criteria:
Death
Life-Threatening: The subject was at substantial risk of dying at the time of the adverse event or it was suspected that the use/continued use of product would result in the subject's death
Hospitalization (initial or prolonged): Required admission to the hospital or prolongation of a hospital stay
Disability: Resulted in a significant, persistent, or permanent change, impairment, damage or disruption in the subject's body function/structure, physical activities or quality of life
Congenital Anomaly/Birth Defect
Important Medical Event: Other medically important events that, in the opinion of the investigator, may have jeopardized the subject, or may have required intervention to prevent one of the other outcomes listed above

A distinction was to be drawn between SAE and severe AE. A severe AE was a major experience of its type. A severe AE was not necessarily serious: e.g. nausea, which persisted for several hours, may have been considered severe nausea, but it was not an SAE. On the other hand a stroke, which resulted in only a limited degree of disability may have been considered a mild stroke, but would have been an SAE.

Timing: All SAEs were reported from the day of initial treatment with the study drug through the completion of the study Day 42. SAEs were captured during a follow-up phone call at Day 42 for subjects who were randomized and terminated early from the trial. Hospitalizations resulting from historical conditions (present prior to initial treatment study drug or prescheduled prior to treatment with study drug) that had not increased in severity or led to prolongation of hospital stay should not have been considered SAEs. All reported serious adverse events should have been followed until they were no longer serious or returned to baseline grade.

Reporting: Any SAE, starting with the first dose of study drug, was to be reported immediately (by the end of the next business day).

In addition to the initial reporting, all SAEs were to be recorded on the Adverse Event page of the CRF and reported immediately to the site's IRB/ethics committee per their reporting guidelines.

The investigator was to determine whether the degree of any untoward event warranted removal of any subject from the study. He/she should have, in any case, instituted appropriate diagnostic and/or therapeutic measures, and kept the subject under observation for as long as was medically indicated.

### Example 8: Statistics

The following example describes the statistical significance of the sample size and statistical analyses.

Stratification/Randomization

Subjects were randomized in a 1:1 ratio within each study site to Injectafer® (iron carboxymaltose) or Placebo (saline). The randomization schedule was prepared before the first subject was enrolled into the study.

Sample Size Rationale

If the proportion of responders (≥13 point improvement in FIQR score on Day 28) is 30% in the placebo group, a sample size of 40 patients per group provided 78% power to detect a 2.0-fold increase in the responder rate (i.e., 30% versus 60%) and >95% power to detect a 2.5-fold increase in the responder rate (i.e., 30% versus 75%).

Disposition and Baseline Characteristics

Disposition was summarized by treatment group. The number and percentage of subjects who were randomized, treated, prematurely discontinued, and completed the study were summarized. The number of subjects in each treatment group and analysis population was summarized.

Subjects with clinically important protocol deviations were identified for each analysis population, treatment group, and type of deviation. The clinical team identified deviations and the deviations were identified in the database.

The number of subjects in each treatment group were summarized for each investigative site. Categorical baseline characteristics were summarized with the number and percent of subjects with the characteristic in each analysis population and treatment group. Quantitative characteristics were summarized with the mean, median, standard deviation, minimum value, and maximum value in each analysis population and treatment group.

Medical history was summarized by treatment group for the Safety Population. Concomitant medications were summarized by treatment group for the Safety Population.

Medical history was coded with the Medical Dictionary for Regulatory Activities (MedDRA) Terminology. The number and percent of subjects with clinically significant medical history at screening were summarized by system organ class (SOC) and preferred term for all subjects.

### Endpoints and Definitions

Primary Endpoints: The primary efficacy variable was the proportion of patients with a ≥13 point improvement in FIQR score on Day 42.

Secondary Endpoints: Other Efficacy Endpoints

1. FIQR scores changed from baseline at each time point and to lowest

2. Brief Pain Inventory total score changed from baseline at each time point

3. MOS Sleep scale total score changed from baseline at each time point

4. Fatigue Visual Numeric Scale changed from baseline at each time point

5. Proportion of subjects required intervention for fibromyalgia and time to fibromyalgia intervention

6. Proportion of subjects with relapse

7. Change from Baseline for iron indices (TSAT, Ferritin, serum iron):

The safety endpoints include:

1. Incidence of treatment emergent adverse events, incidence of serious adverse events, and incidence of severe adverse events

2. Change in clinical laboratory tests

3. Change in vital signs

The Safety Population consisted of all subjects who received at least 1 dose of randomized treatment. All safety analyses were performed with the Safety Population. The Efficacy Evaluable Population consisted of all subjects who received at least one dose of randomized treatment with at least one completed post treatment FIQR evaluation. All efficacy analyses were performed with the Efficacy Evaluable Population.

Treatment compliance was summarized as the volume (mL) of doses administered for each dose day and the percent of planned volume received (100%×the number planned/the number received). Descriptive statistics included the mean, standard deviation, median, and minimum and maximum values. Compliance was summarized by treatment group for the Safety and Efficacy Evaluable Populations.

Statistical Analyses of Efficacy

Categorical variables were summarized with the number and percent of subjects in each treatment group. Quantitative variables were summarized with the mean, median, standard deviation, minimum value, and maximum value. Baseline was defined as the last value obtained before randomized treatment.

Treatment group differences for proportions were assessed with the chi-square test. Treatment group differences for means were assessed with the analysis of covariance with treatment and study site as fixed factors and with baseline score as a covariate.

The mean difference between groups and the 95% confidence interval for the mean difference were summarized. The 95% confidence interval was calculated from the analysis of covariance with fixed factor for treatment and baseline score as a covariate.

A within-group correlation analysis compared the baseline IRLS score to overall improvement in the FIQR score on Day 42 using the Pearson R or a Spearman rank method, as appropriate.

Time to intervention was to be analyzed using the log-rank test.

For the purpose of the primary efficacy analysis, subjects who discontinued or did not complete the study were considered as non-responders. For comparison of means, the primary imputation method was the last observation carried forward. Sensitivity analyses assessed the impact of missing values on inferences by conducting the primary analysis with and without the imputation. All statistical tests were performed with a Type I error of 0.05, 2-tailed.

Statistical Analyses of Safety

Analyses of safety data were descriptive and no formal statistical comparisons were made.

The Medical Dictionary for Regulatory Activities (MedDRA) Terminology was used to classify all adverse events with respect to system organ class and preferred term. The number and proportion of subjects who report treatment-emergent adverse events were summarized from each treatment group. A similar summary was provided for all treatment emergent serious adverse events.

The adverse event profile was characterized with severity (as graded by Version 4.0 of the National Cancer Institute Common Terminology Criteria for Adverse Events [NCI-CTCAE]) and relationship to study drug. Relationship to study drug was categorized as related (possibly or probably related) and unrelated (none or unlikely). Adverse events with unknown severity or relationship were counted as unknown.

Subjects who reported the same preferred term on multiple occasions were counted once for the preferred term: under the highest severity when summarized by severity and under the closest relationship to study drug when summarized by relationship. If a subject reported multiple preferred terms for a system organ class (SOC), the subject was counted only once for that SOC.

Changes in clinical laboratory findings and vital signs from baseline to each scheduled study visit were summarized descriptively with the mean, median, standard deviation, minimum value, and maximum value. The number and percent of patients with potentially clinically significant clinical laboratory values and vital signs were summarized for each treatment group.

Treatment-emergent PCS laboratory tests were defined as those for which the baseline value was normal and post-baseline value was abnormal (i.e., met Grade 3 or Grade 4 toxicity criteria from the NCI-CTCAE). Subjects with PCS values were identified.

Treatment emergent PCS vital signs were identified with the criteria in TABLE 3 and summarized descriptively for each treatment group.

TABLE 3
Criteria for Potentially Clinically Significant (PCS) Vital Signs

| | | Definition of PCS at immediately and 30 |
|---|---|---|
| Vital Sign | Criterion | minutes post-dose |
| Systolic | Low | Value ≤90 mmHg and decreased ≥20 mmHg |
| blood | | from pre dose^{a} value |
| pressure | High | Value ≥80 mmHg and increased ≥20 mmHg |
| | | from pre dose^{a} value |
| Diastolic | Low | Value ≤50 mmHg and decreased ≥15 mmHg |
| blood | | from pre dose^{a} value |
| pressure | High | Value ≥105 mmHg and increased ≥15 mmHg |
| | | from pre-dose^{a} value |
| Pulse | Low | Value ≤50 bpm and decreased ≥15 bpm from |
| | | pre-dose^{a} value |
| | High | Value ≥100 bpm and increased ≥15 bpm |
| | | from pre-dose^{a} value |

^{a}The pre-dose value was the value obtained prior to dosing on each day of dosing.

The efficacy and safety measurements evaluated in this study were standard and accepted routine medical practice.

This study was performed in accordance with FDA and ICH Good Clinical Practices, 21CFR Part 312.

Calculation of Efficacy Scores

Revised Fibromyalgia Impact Questionnaire (FIQR).

The FIQR was divided into 3 domains: Function, Overall Impact, and Symptoms. Each domain was calculated on a cluster of items in the questionnaire, with each item graded on a 0 to 10 numeric rating scale. A lower score is associated with a better quality of life. Function domain score=sum of point values from items 1-9. Overall Impact domain score=sum of point values from items 10-11. Symptoms domain score=sum of point values from items 12-21.

If 3 or more of the questions were unanswered (missing) then the questionnaire was to be considered invalid and the total score set to null. In the event of missing responses (<3), weighting was applied in calculating domain scores. The added score of the completed questions was weighted by the total number of items in the domain divided by the number completed in the domain. For example, if X items were completed, the weighting value for Function would have been 9/X, 2/X and 10/X for the Function, Overall Impact, and Symptoms domain scores, respectively.

The total FIQR score was calculated from the 3 normalized domain scores, as shown below:

(Function domain score/3)+(Overall Impact domain score/1)+(Symptoms Domain Score/2)

Brief Pain Inventory.

The Brief Pain Inventory short form measures the 2 domains of Pain Severity and Pain Interference. Pain Severity was based on the added scores from items 3, 4, 5, and 6 and Pain Interference was based on the added scores from items 9a through 9g. Each of these items was graded on a 0 to 10 numeric rating scale. A higher score was associated with greater Pain Severity and interference. The Brief Pain Inventory total score was calculated from the sum of both domains: Sum of scores from items 3,4,5,6+Sum of scores from items 9a through 9g.

Question 2 on the Brief Pain Inventory was an anatomical diagram where the subject put X's on all the areas that hurt. No data were entered from the question.

Medical Outcomes Study (MOS) Sleep.

The 6-item version of the MOS Sleep Scale comprises 6 items from the 12-item version: 2 items from each of the Sleep Disturbance (items C and D) and Sleep Adequacy (items A and F) subscales and 1 item from each of the Shortness of Breath or Headache (item B) and Sleep Somnolence (item E) subscales. The Sleep Scale total score is the sum of all 6 items.

Fatigue Visual Numeric Scale.

The Fatigue Visual Numeric score was the subject-completed number circled or histogram marked value. Scores ranged from 0 to 10, with higher scores indicating more fatigue.

International Restless Legs Syndrome Group Rating Scale (IRLS).

The IRLS was composed of 10 items. Nine of the 10 items investigate 2 dimensions. The symptoms dimension was the sum of 6 items: 1, 2, 4, 6, 7, and 8 with a higher score equated with higher severity; and the Symptoms Impact dimension was the sum of 3 items: 5, 9 and 10 with a higher score equated with higher impact. The sum of the 2 dimensions plus item 3 was used to calculate the overall severity score (total score). All 10 items should have been completed to calculate the total score; there was no weighting of scores.

The IRLS was administered only at baseline and was used as a potential explanatory factor for observed changes from baseline in FIQR.

### Example 9: Study Subjects

The following example describes the study subjects.

Subject Disposition

A total of 81 subjects (41 Injectafer®, 40 placebo) were randomized and treated with at least 1 dose of study drug. Two subjects (1 Injectafer®, 1 placebo) were discontinued from the study. No subject required an intervention for intolerable fibromyalgia or became pregnant while on study. A summary of subject disposition and study termination is presented in TABLE 4.

TABLE 4
Subject Disposition (All Randomized Subjects)

| | Group A | | Group B: | |
|---|---|---|---|---|
| | Injectafer ® | | Placebo | |
| Subjects randomized | 41 | | 40 | |
| Subjects treated | 41 | | 40 | |
| Subjects completed | 40 | (97.6) | 39 | (97.5) |

| | | | |
|---|---|---|---|
| Subjects discontinued | | | |
| Lost to follow-up | 1 | (2.4) | 0 |

| | | | |
|---|---|---|---|
| Other | 0 | 1 | (2.5)^{a} |

| | | |
|---|---|---|
| Intervention for intolerable fibromyalgia | 0 | 0 |
| Became pregnant on study | 0 | 0 |

^{a}Difficult phlebotomy during study drug administration on Day 5.

All subjects were enrolled at a single site.

Protocol Deviations

One randomized subject in the Injectafer® group (Subject 8001-104) did not meet exclusion criterion 16 (calcium or phosphorous outside the normal range). The subject's phosphorous value at screening was 5.6 mg/dL (normal range: 2.5 to 5.0 mg/dL).

One subject in the placebo group (Subject 8001-125) had a difficult phlebotomy during study drug administration on Day 5 and was discontinued.

No other protocol deviations were identified.

### Example 10: Efficacy Evaluation

The following example describes the efficacy evaluation of the study.

Data Sets Analyzed

No subjects were excluded from the Safety Population. One subject (8001-104) in the Injectafer® group was excluded from the Efficacy Evaluable Population. The subject had no post-baseline FIQR completed.

Demographic and Other Baseline Characteristics

Demographic characteristics.

No clinically significant differences were observed between the Injectafer® and placebo groups in the Safety Population for any of the demographic characteristics. Among all subjects, mean age was 42.5 years and 98.8% of subjects were female. The most common races were Caucasian (77.8%) and African-American (17.3%). The mean IRLS score was 14.8.

A history of iron intolerance was infrequent (11.1% of subjects) and due to ferrous sulfate. Symptoms of intolerance to ferrous sulfate included nausea and constipation. Approximately half of subjects (46.9%) reported a history of drug allergies.

A summary of the demographic characteristics of the Injectafer® and placebo groups is presented in TABLE 5.

TABLE 5
Demographic Characteristics at Baseline (Safety Population)

| | Group A: | | Group B: | | | |
|---|---|---|---|---|---|---|
| | Injectafer ® | | Placebo | | Total | |
| | (N = 40) | | (N = 40) | | (N = 81) | |
| Age (years) | | | | | | |
| Mean (SD) | 41.2 | (11.07) | 43.9 | (10.84) | 42.5 | (10.97) |
| Median | 39.2 | | 44.9 | | 42.6 | |
| Minimum, | 22.3, 68.4 | | 18.8, 65.2 | | 18.8, 68.4 | |
| maximum | | | | | | |
| Sex, n (%) | | | | | | |
| Female | 40 | (97.6) | 40 | (100.0) | 80 | (98.8) |
| Male | 1 | (2.4) | 0 | | 1 | (1.2) |
| Race, n (%) | | | | | | |
| African American | 7 | (17.1) | 7 | (17.5) | 14 | (17.3) |
| Asian | 1 | (2.4) | 0 | | 1 | (1.2) |
| Caucasian | 33 | (80.5) | 30 | (75.0) | 63 | (77.8) |
| Hispanic | 0 | | 2 | (5.0) | 2 | (2.5) |
| Other | 0 | | 1 | (2.5) | 1 | (1.2) |
| History of iron | | | | | | |
| tolerance | | | | | | |
| No | 35 | (85.4) | 37 | (92.5) | 72 | (88.9) |
| Yes^{a} | 6 | (14.6) | 3 | (7.5) | 9 | (11.1) |
| History of drug | | | | | | |
| allergy | | | | | | |
| No | 21 | (51.2) | 17 | (42.5) | 38 | (46.9) |
| Yes | 20 | (48.8) | 23 | (57.5) | 43 | (53.1) |
| IRLS score (kg) | | | | | | |
| Mean (SD) | 13.4 | (13.16) | 16.3 | (14.90) | 14.8 | (14.04) |

| | | | |
|---|---|---|---|
| Median | 18.0 | 22.5 | 18.0 |
| Minimum, | 0.0, 35.0 | 0.0, 38.0 | 0.0, 38.0 |
| maximum | | | |

^{a}Intolerance to ferrous sulfate was indicated for the 9 subjects

Medical History.

No clinically significant differences were observed between the Injectafer® and placebo groups in the Safety Population for medical history. In addition to fibromyalgia reported for all subjects, the most common (≥30.0% of all subjects) baseline medical histories included depression (53.1%), hypertension (43.2%), iron-deficiency anemia (39.5%), anxiety (35.8%), and tubal ligation (34.6%).

Concomitant Medications.

The types of medications received during the study were generally similar between the groups. The most common (≥10.0% of all subjects) concomitant medications included ibuprofen (29.6%), Tylenol (22.2%), phentermine (18.5%), Aleve (16.0%), metformin (14.8%), Lisinopril (12.3%), Advil (11.1%), and vitamin D (11.1%). Additionally, use of ferrous sulfate was reported for 17.3% of subjects; per protocol, its use was discontinued at screening.

Measurement of Treatment Compliance.

One subject in the placebo group (Subject 8001-125) had a difficult phlebotomy during study drug administration on Day 5 and was discontinued. The other subjects received 100% to 100.2% of both planned doses. Starting and stopping times of Injectafer® and placebo administration and the total doses administered were documented. A summary of the number of study drug doses received and the percentage of planned dose received for the Injectafer® and placebo groups is presented in TABLE 6.

TABLE 6
Summary of Number of Study Drug Doses Received and Percentage of
Planned Dose Received (Safety Population)

| | Group A: | | Group B: | |
|---|---|---|---|---|
| | Injectafer ® | | Placebo | |
| | (N = 41) | | (N = 40) | |
| Total number of doses received, n (%) | | | | |
| 1 dose | 0 | | 1 | (2.5) |
| 2 doses | 41 | (100.0) | 39 | (97.5) |
| Percentage of planned doses received | | | | |
| (Dose 1) | | | | |
| Number of subjects | 41 | | 40 | |
| Mean (SD) | 100.0 | (0.00) | 100.0 | (0.03) |
| Median | 100.0 | | 100.0 | |
| Minimum, maximum | 100.0, 100.0 | | 100.0, 100.2 | |
| Percentage of planned doses received | | | | |
| (Dose 2) | | | | |
| Number of subjects | 41 | | 39 | |
| Mean (SD) | 100.0 | (0.00) | 100.0 | (0.03) |

| | | |
|---|---|---|
| Median | 100.0 | 100.0 |
| Minimum, maximum | 100.0, 100.0 | 100.0, 100.2 |

Efficacy Results and Tabulations of Individual Subject Data

Analysis of Efficacy.

(i) Revised Fibromyalgia Impact Questionnaire (FIQR) The percentage of subjects who had a ≥3-point improvement in the FIQR from baseline to Day 42 was greater in the Injectafer® group than the placebo group (76.9% versus 66.7%). However, the difference was not statistically significant (p=0.314). The proportion of subjects with a ≥3-point improvement in the FIQR from baseline to Day 42 is summarized in TABLE 7.

TABLE 7
Proportion of Subjects with ≥13-Point Improvement in FIQR From
Baseline to Day 42 (Efficacy Evaluable Population)

| | Group A: | Group B: |
|---|---|---|
| | Injectafer ® | Placebo |
| | (N = 40) | (N = 40) |
| Achieved ≥13 point improvement, n/N (%) | 30/39 (76.9) | 26/39 (66.7) |

| | |
|---|---|
| Difference (95% confidence interval) | 0.103 (−0.122, 0.327) |
| Fisher's exact p-value | 0.314 |

Statistically significantly greater improvement from baseline in FIQR total score was observed for the Injectafer® group as compared to the placebo group on Days 14, 28, and 42. Mean improvement from baseline in FIQR total score increased in the Injectafer® group from 33.8 on Day 14 to 45.0 on Day 42. Mean improvement at the lowest post-baseline value was −51.6 in the Injectafer® group and −31.9 in the placebo group (p<0.001). The total score and change from baseline for FIQR by visit are summarized in TABLE 8.

TABLE 8
Total Score and Change from Baseline for FIQR by Visit
(Efficacy Evaluable Population)

| | | | | | Difference | |
|---|---|---|---|---|---|---|
| | Group A: | | Group B: | | 95% | |
| Visit | Injectafer ® | | Placebo | | Confidence | p- |
| Statistic | (N = 40) | | (N = 40) | | Interval | value^{a} |
| Baseline | | | | | | |
| N | 40 | | 40 | | | |
| Mean | 82.8 | (9.45) | 83.1 | (9.43) | | |
| Day 14 | | | | | | |
| N | 40 | | 39 | | | |
| Mean (SD) | 49.0 | (26.99) | 66.1 | (19.99) | | |
| Mean change | −33.8 | (24.85) | −17.1 | (17.67) | −16.62 | 0.001 |
| (SD) | | | | | (−26.4, −6.9) | |
| Day 28 | | | | | | |
| N | 40 | | 39 | | | |
| Mean (SD) | 37.6 | (27.86) | 58.2 | (23.08) | | |
| Mean change | −45.2 | (26.70) | −25.0 | (21.85) | −20.21 | <0.001 |
| (SD) | | | | | (−31.2, −9.2) | |
| Day 42 | | | | | | |
| N | 38 | | 39 | | | |
| Mean (SD) | 37.6 | (30.11) | 53.6 | (28.68) | | |
| Mean change | −45.0 | (29.46) | −29.7 | (26.97) | −12.41 | 0.015 |
| (SD) | | | | | (−22.3, −2.5) | |
| Lowest post- | | | | | | |
| baseline value | | | | | | |
| N | 40 | | 40 | | | |
| Mean change | −51.6 | (24.42) | −31.9 | (25.47) | −19.73 | <0.001 |

| | |
|---|---|
| (SD) | (−30.9, −8.6) |

^{a}Treatment group differences were assessed using analysis of covariance with a fixed factor for treatment and baseline score as a covariate.

Results were similar when missing values were imputed.

The correlation between baseline IRLS score and change in FIQR from baseline to Day 42 was not statistically significant within either treatment group. The correlation between baseline IRLS score and change in FIQR from baseline to Day 42 is summarized in TABLE 9.

TABLE 9
Correlation Between baseline IRLS Score and Change in FIQR from
Baseline to Day
42 (Efficacy Evaluable Population)

| | Group A: | | Group B: | |
|---|---|---|---|---|
| Visit | Injectafer ® | | Placebo | |
| Statistic | (N = 40) | | (N = 40) | |
| Baseline IRLS score | | | | |
| N | 40 | | 40 | |
| Mean (SD) | 12.9 | (13.02) | 16.3 | (14.90) |
| Day 42 FIQR score | | | | |
| N | 38 | | 39 | |
| Mean (SD) | −45.0 | (29.46) | −29.7 | (26.97) |

| | | |
|---|---|---|
| Correlation coefficient for baseline | −0.162 | 0.001 |
| IRLS score with Day 42 FIQR score | | |
| p-value | 0.331 | 0.994 |

(ii) Brief Pain Inventory

Statistically significantly greater improvement from baseline in Brief Pain Inventory Pain Severity score was observed for the Injectafer® group as compared to the placebo group on Days 14, 28, and 42. Mean improvement from baseline in Brief Pain Inventory Pain Severity score increased in the Injectafer® group from −2.3 on Day 14 to −3.1 on Day 42.

Statistically significantly greater improvement from baseline in Brief Pain Inventory Pain Interference score was observed for the Injectafer® group as compared to the placebo group on Days 14, 28, and 42. Mean improvement from baseline in Brief Pain Inventory Pain Interference score in the Injectafer® group increased from −3.2 on Day 14 to −4.1 on Day 42.

The total score and change from baseline for Brief Pain Inventory Pain Severity and Pain Interference scores by visit are summarized in TABLE 10.

TABLE 10
Mean Total Score and Mean Change from Baseline for Brief Pain
Inventory by Visit
(Efficacy Evaluable Population)

| | | | | |
|---|---|---|---|---|
| | | | Difference | |
| | Group A: | Group B: | 95% | |
| Visit | Injectafer ® | Placebo | Confidence | p- |
| Statistic | (N = 40) | (N = 40) | Interval | value^{a} |

Pain Severity Scale

| Baseline | | | | | | |
|---|---|---|---|---|---|---|
| N | 40 | | 40 | | | |
| Mean (SD) | 6.9 | (1.23) | 7.0 | (1.40) | | |
| Day 14 | | | | | | |
| N | 40 | | 39 | | | |
| Mean (SD) | 4.6 | (2.29) | 6.1 | (2.03) | | |
| Mean change | −2.3 | (2.23) | −0.9 | (1.18) | −1.44 | <0.001 |
| (SD) | | | | | (−2.2, −0.6) | |
| Day 28 | | | | | | |
| N | 40 | | 39 | | | |
| Mean (SD) | 4.0 | (2.59) | 5.7 | (2.27) | | |
| Mean change | −3.0 | (2.41) | −1.3 | (1.82) | −1.70 | <0.001 |
| (SD) | | | | | (−2.7, −0.7) | |
| Day 42 | | | | | | |
| N | 38 | | 39 | | | |
| Mean (SD) | 3.8 | (2.71) | 5.2 | (2.56) | | |
| Mean change | −3.1 | (2.69) | −1.8 | (2.13) | −1.29 | 0.022 |

| | |
|---|---|
| (SD) | (−2.4, −0.2) |

Pain Interference Scale

| Baseline | | | | | | |
|---|---|---|---|---|---|---|
| N | 40 | | 40 | | | |
| Mean (SD) | 8.1 | (1.22) | 7.9 | (1.34) | | |
| Day 14 | | | | | | |
| N | 40 | | 39 | | | |
| Mean (SD) | 4.9 | (2.86) | 6.9 | (2.07) | | |
| Mean change | −3.2 | (2.81) | −1.0 | (2.00) | −2.10 | <0.001 |
| (SD) | | | | | (−3.2, −1.0) | |
| Day 28 | | | | | | |
| N | 40 | | 39 | | | |
| Mean (SD) | 4.0 | (3.15) | 6.2 | (2.49) | | |
| Mean change | −4.1 | (3.21) | −1.8 | (2.41) | −2.25 | <0.001 |
| (SD) | | | | | (−3.5, −1.0) | |
| Day 42 | | | | | | |
| N | 38 | | 39 | | | |
| Mean (SD) | 3.9 | (3.13) | 5.6 | (2.81) | | |
| Mean change | −4.1 | (3.29) | −2.3 | (2.55) | −1.74 | 0.010 |

| | |
|---|---|
| (SD) | (−3.1, −0.4) |

^{a}Treatment group differences were assessed using analysis of covariance with a fixed factor for treatment and baseline score as a covariate.

Results were similar when missing values were imputed for the Pain Severity score and Pain Interference score.

(iii) Medical Outcomes Study (MOS) Sleep Scale

Mean change in the MOS Sleep Scale was similar for the 2 treatment groups at each visit and no statistically significant treatment group differences were observed.

The MOS Sleep Scale score and change from baseline by visit are summarized in

TABLE 11
Medical Outcomes Study Sleep Scale Score and Change from
Baseline by Visit (Efficacy Evaluable Population)

| | | | | | Difference | |
|---|---|---|---|---|---|---|
| | Group A: | | Group B: | | 95% | |
| Visit | Injectafer ® | | Placebo | | Confidence | p- |
| Statistic | (N = 40) | | (N = 40) | | Interval | value^{a} |
| Baseline | | | | | | |
| N | 39 | | 40 | | | |
| Mean | 72.7 | (13.13) | 79.5 | (12.44) | | |
| Day 14 | | | | | | |
| N | 40 | | 39 | | | |
| Mean (SD) | 63.2 | (16.12) | 66.1 | (19.99) | | |
| Change | | | | | | |
| N | 39 | | 39 | | | |
| Mean Change | −9.2 | (16.30) | −6.6 | (14.36) | −5.58 | 0.104 |
| (SD) | | | | | (−12.3, 1.2) | |
| Day 28 | | | | | | |
| N | 39 | | 39 | | | |
| Mean (SD) | 60.4 | (17.02) | 70.1 | (16.34) | | |
| Change | | | | | | |
| N | 38 | | 39 | | | |
| Mean change | −12.1 | (18.99) | −9.6 | (14.91) | −6.12 | 0.102 |
| (SD) | | | | | (−13.5, 1.2) | |
| Day 42 | | | | | | |
| N | 38 | | 37 | | | |
| Mean (SD) | 59.5 | (17.77) | 70.2 | (17.86) | | |
| Change | | | | | | |
| N | 37 | | 37 | | | |
| Mean change | −13.6 | (18.03) | −9.5 | (17.14) | −6.98 | 0.083 |

| | |
|---|---|
| (SD) | (−14.9, 0.9) |

^{a}Treatment group differences were assessed using analysis of covariance with a fixed factor for treatment and baseline score as a covariate.

Results were similar when missing values were imputed.

(iv) Fatigue Visual Numeric Scale

Statistically significantly greater improvement from baseline in the Fatigue Visual Numeric Scale was observed for the Injectafer® group as compared to the placebo group on Days 14, 28, and 42. Mean improvement from baseline in the Fatigue Visual Numeric Scale increased in the Injectafer® group from −2.5 on Day 14 to −3.7 on Day 42. The total score and change from baseline for the Fatigue Visual Numeric Scale by visit are summarized in TABLE 12.

TABLE 12
Mean Total Score and Mean Change from Baseline for Fatigue Visual
Numeric Scale by Visit
(Efficacy Evaluable Population)

| | Group A: | | Group B: | | Difference | |
|---|---|---|---|---|---|---|
| Visit | Injectafer ® | | Placebo | | 95% Confidence | p- |
| Statistic | (N = 40) | | (N = 40) | | Interval | value^{a} |
| Baseline | | | | | | |
| N | 40 | | 40 | | | |
| Mean | 9.2 | (1.0) | 9.3 | (0.92) | | |
| Day 14 | | | | | | |
| N | 40 | | 39 | | | |
| Mean (SD) | 6.6 | (2.38) | 8.0 | (1.57) | | |
| Mean change | −2.5 | (2.55) | −1.3 | (1.52) | −1.33 | 0.005 |
| (SD) | | | | | (−2.2, −0.4) | |
| Day 28 | | | | | | |
| N | 40 | | 39 | | | |
| Mean (SD) | 6.0 | (2.86) | 7.7 | (2.07) | | |
| Mean change | −3.2 | (3.04) | −1.6 | (1.98) | −1.62 | 0.005 |
| (SD) | | | | | (−2.7, −0.5) | |
| Day 42 | | | | | | |
| N | 37 | | 39 | | | |
| Mean (SD) | 5.4 | (2.93) | 7.6 | (2.28) | | |
| Mean change | −3.7 | (3.21) | −1.7 | (2.20) | −2.18 | <0.001 |

| | |
|---|---|
| (SD) | (−3.4, −1.0) |

^{a}Treatment group differences were assessed using analysis of covariance with a fixed factor for treatment and baseline score as a covariate.

Results were similar when missing values were imputed.

(v) Intervention for Fibromyalgia

No subject required intervention for fibromyalgia.

(vi) Iron Indices

Statistically significantly greater mean increases from baseline to Day 42 in serum ferritin, iron, and TSAT were observed for the Injectafer® group as compared to the placebo group. A statistically significantly greater mean decrease from baseline to Day 42 in TIBC was observed for the Injectafer® group as compared to the placebo group. Baseline and change from baseline to Day 42 for iron indices are summarized in TABLE 13.

TABLE 13
Baseline and Change from Baseline to Day 42 for Iron Indices
(Efficacy Evaluable Population)

| | | | |
|---|---|---|---|
| | Group A: | Group B: | |
| Visit | Injectafer ® | Placebo | |
| Statistic | (N = 40) | (N = 40) | p-value^{a} |

Serum Ferritin (μg/L)

| Screening | | | | | |
|---|---|---|---|---|---|
| N | 40 | | 40 | | |
| Mean (SD) | 19.0 | (12.37) | 18.1 | (11.59) | |
| Day 42 | | | | | |
| N | 39 | | 38 | | |
| Mean (SD) | 311.3 | (152.10) | 20.0 | (17.41) | |
| Mean change (SD) | 292.5 | (145.86) | 2.0 | (13.78) | <0.001 |

Serum Iron (μmol/L)

| Screening | | | | | |
|---|---|---|---|---|---|
| N | 40 | | 40 | | |
| Mean (SD) | 52.2 | (20.18) | 51.8 | (21.16) | |
| Day 42 | | | | | |
| N | 39 | | 38 | | |
| Mean (SD) | 105.1 | (37.72) | 69.5 | (61.08) | |
| Mean change (SD) | 53.2 | (37.21) | 18.4 | (63.55) | <0.001 |

Total Iron Binding Capacity (μmol/L)

| Screening | | | | | |
|---|---|---|---|---|---|
| N | 40 | | 40 | | |
| Mean (SD) | 371.4 | (48.39) | 387.8 | (50.58) | |
| Day 42 | | | | | |
| N | 39 | | 38 | | |
| Mean (SD) | 272.4 | (46.29) | 377.9 | (78.87) | |
| Mean change (SD) | −100.3 | (49.75) | −9.0 | (50.82) | <0.001 |

Transferrin Saturation (%)

| Screening | | | | | |
|---|---|---|---|---|---|
| N | 40 | | 40 | | |
| Mean (SD) | 14.6 | (5.25) | 13.6 | (5.54) | |
| Day 42 | | | | | |
| N | 39 | | 38 | | |
| Mean (SD) | 38.9 | (11.96) | 19.3 | (15.39) | |
| Mean change (SD) | 24.4 | (11.39) | 5.8 | (15.07) | <0.001 |

^{a}Treatment group differences were assessed using analysis of covariance with a fixed factor for treatment and baseline value as a covariate.

Statistical/Analytical Issues.

(i) Adjustments for Covariates

The baseline value was used as a covariate in the analysis of covariance for change from baseline.

(ii) Handling of Dropouts or Missing Data

For the primary efficacy endpoint, subjects who discontinued from the study were considered non responders. Missing data were imputed with last observation carried forward as a sensitivity analysis for secondary efficacy endpoints.

(iii) Interim Analyses and Data Monitoring

No formal interim analysis was planned or performed in this study.

(iv) Multicenter Studies

This was a single center study.

(v) Multiple Comparisons/Multiplicity

No adjustments for multiple comparisons/multiplicity were performed.

(vi) Use of an Efficacy Subset of Subjects

All efficacy analyses were performed with the Efficacy Evaluable Population, which consisted of all subjects who received at least 1 dose of randomized treatment and had at least 1 completed post-treatment FIQR evaluation.

Efficacy Conclusions.

The primary efficacy endpoint was the percentage of subjects who had a ≥3-point improvement in the FIQR from baseline to Day 42. The proportion of subjects achieving the primary endpoint was greater in the Injectafer® group than the placebo group (76.9% versus 66.7%). However, the difference was not statistically significant (p=0.314).

Key findings for secondary efficacy endpoints include:
Statistically significantly greater improvement from baseline in FIQR total score was observed for the Injectafer® group as compared to the placebo group on Days 14, 28, and 42. Mean improvement from baseline in FIQR total score increased in the Injectafer® group from 33.8 on Day 14 to 45.0 on Day 42. Mean improvement at the lowest post baseline value was −51.6 in the Injectafer® group and −31.9 in the placebo group (p<0.001).
Statistically significantly greater improvement from baseline in both the Brief Pain Inventory Pain Severity and Pain Interference scores was observed for the Injectafer® group as compared to the placebo group on Days 14, 28, and 42.
Mean change in the MOS Sleep Scale was similar for the 2 treatment groups at each visit and no statistically significant differences were observed.
Statistically significantly greater improvement from baseline in the Fatigue Visual Numeric Scale was observed for the Injectafer® group as compared to the placebo group on Days 14, 28, and 42.
Statistically significantly greater mean increases from baseline to Day 42 in serum ferritin, iron, and TSAT were observed for the Injectafer® group as compared to the placebo group. A statistically significantly greater mean decrease from baseline to Day 42 in TIBC was observed for the Injectafer® group as compared to the placebo group.

In summary, statistically significantly greater mean improvement was observed for Injectafer® as compared to placebo for FIQR total score, Brief Pain Inventory Pain Severity and Pain Interference scores, Fatigue Visual Numeric Scale, and iron indices.

### Example 11: Safety Evaluation

The following example describes the safety evaluation of the study. All safety analyses were performed using the Safety Population, which consisted of all subjects who received at least 1 dose of randomized treatment.

Extent of Exposure

One subject in the placebo group (Subject 8001-125) had a difficult phlebotomy during study drug administration on Day 5 and was discontinued. The other subjects received at least 100% to 100.2% of both planned doses.

Starting and stopping times of Injectafer® and placebo administration and the total doses administered were documented. A summary of the number of study drug doses received and the percentage of planned dose received for the Injectafer® and placebo groups is presented in TABLE 6, above.

Adverse Events

Brief Summary of Adverse Events

A larger percentage of subjects in the Injectafer® group (29.3%) than the placebo group (5.0%) reported at least 1 treatment-emergent adverse event. At least 1 drug-related treatment emergent adverse event was reported by 24.4% of subjects in the Injectafer® group and no subjects in the placebo group. There were no treatment-emergent serious adverse events and no treatment-emergent adverse event led to discontinuation of study drug. An overview of treatment-emergent adverse events is presented in TABLE 14.

TABLE 14
Overview of Treatment-Emergent Adverse Events (Safety Population)

| | Group A: | Group B: |
|---|---|---|
| | Injectafer ® | Placebo |
| | (N = 40) | (N = 40) |
| Type of Treatment-Emergent Adverse Event^{a} | n (%) | n (%) |
| Any | 12 (29.3) | 2 (5.0) |
| At least 1 serious | 0 | 0 |
| At least 1 severe^{b} | 0 | 0 |
| At least 1 drug-related^{c} | 10 (24.4) | 0 |
| At least 1 leading to discontinuation of | 0 | 0 |
| study drug | | |

^{a}If a subject experienced the same event more than once, the first occurrence was tabulated.
^{b}Grade 3, 4, or 5.
^{c}Possibly or probably related to study drug.
Display and Analyses of Adverse Events
All Treatment Emergent Adverse Events.

During the study, at least 1 treatment emergent adverse event was experienced by 29.3% (12/41) of the subjects in the Injectafer® group and 5.0% (2/40) of the subjects in the placebo group. Treatment emergent adverse events reported by ≥2 subjects in the Injectafer® group were nausea (7.3%), flushing (14.6%), and dizziness (4.9%). No treatment emergent adverse events were reported by ≥2 subjects in the placebo group. All treatment-emergent adverse events were mild in severity. A summary of treatment emergent adverse events experienced by ≥2 subjects in either the Injectafer® or placebo group during the study is presented in TABLE 15.

TABLE 15
Treatment-Emergent Adverse Events Experienced by ≥2 Subjects in
Either the Injectafer ® or Placebo Group (Safety Population)

| | Group A: | | Group B: | |
|---|---|---|---|---|
| | Injectafer ® | | Placebo | |
| | (N = 40) | | (N = 40) | |
| MedDRA System Organ Class^{a} | n (%) | | n (%) | |
| At Least 1 Treatment-Emergent Adverse Event | 12 | (29.3) | 2 | (5.0) |

| | | | |
|---|---|---|---|
| Gastrointestinal Disorders | 3 | (7.3) | 0 |
| Nausea | 3 | (7.3) | 0 |
| Nervous System Disorders | 5 | (12.2) | 0 |
| Dizziness | 2 | (4.9) | 0 |
| Vascular Disorders | 6 | (14.6) | 0 |
| Flushing | 6 | (14.6) | 0 |

^{a}Each subject is counted only once per system organ class when multiple preferred terms are reported for the system organ class.

Drug-Related Treatment-Emergent Adverse Events.

During the study, at least 1 drug related treatment emergent adverse event (defined as possibly or probably related) was experienced by 24.4% (10/41) of the subjects in the Injectafer® group and no subjects in the placebo group. Drug related treatment emergent adverse events experienced by ≥2 subjects in the Injectafer® group were flushing (12.2%), dizziness (4.9%), and nausea (4.9%). A summary of drug related treatment emergent adverse events experienced during the study is presented in TABLE 16.

TABLE 16
Drug-Related Treatment-Emergent Adverse Events Experienced
During the Study (Safety Population)

| | Group A: | | Group B: |
|---|---|---|---|
| | Injectafer ® | | Placebo |
| MedDRA System Organ Class^{a} | (N = 40) | | (N = 40) |
| Preferred Term | n (%) | | n (%) |
| At Least 1 Treatment-Emergent Adverse Event | 10 | (24.4) | 0 |
| Ear and Labyrinth Disorders | 1 | (2.4) | 0 |
| Ear discomfort | 1 | (2.4) | 0 |
| Gastrointestinal Disorders | 2 | (4.9) | 0 |
| Nausea | 2 | (4.9) | 0 |
| Nervous System Disorders | 5 | (12.2) | 0 |
| Dizziness | 2 | (4.9) | 0 |
| Dysgeusia | 1 | (2.4) | 0 |
| Headache | 1 | (2.4) | 0 |
| Paraesthesia | 1 | (2.4) | 0 |
| Vascular Disorders | 5 | (12.2) | 0 |
| Flushing | 5 | (12.2) | 0 |

^{a}Each subject is counted only once per system organ class when multiple preferred terms are reported for the system organ class.

Severity.

All treatment-emergent adverse events were mild in severity.

Deaths, Other Serious Adverse Events, and Other Significant Adverse Events

No subjects died during the study. No subjects reported treatment emergent serious adverse events during the study. No treatment emergent adverse event led to discontinuation of study drug. No subjects reported a treatment-emergent adverse event of hypersensitivity, allergic reaction, hypotension, or hypertension, during the study. All pregnancy test results in the study were negative. No subjects died, experienced treatment-emergent serious adverse events, or discontinued study drug due to treatment-emergent adverse events during the study. No subjects reported a treatment-emergent adverse event of hypersensitivity, allergic reaction, hypotension, or hypertension, during the study.

Evaluation of Each Laboratory Parameter

Hematology.

Mean increases from screening to Day 42 were observed for hematocrit, hemoglobin, MCH, MCHC, RBC, and RDW in the Injectafer® group compared to mean decreases in the placebo group. A greater mean increase in MCV from screening to Day 42 was observed for subjects in the Injectafer® group compared to subjects in the placebo group. A greater mean decrease in platelets from screening to Day 42 was observed for subjects in the Injectafer® group compared to subjects in the placebo group. The mean change in hemoglobin from baseline to Day 42 was 1.2 g/dL in the Injectafer group and −0.1 g/dL in the placebo group. The mean hemoglobin value at baseline was 12.3 g/dL in both treatment groups. A summary of mean changes from screening to Day 42 in hematology parameters is presented in TABLE 17.

TABLE 17
Mean Changes From Screening to Day 42 in Hematology Parameters (Safety Population)

| | Injectafer ® | | | | | | Placebo | | | | | |
|---|---|---|---|---|---|---|---|---|---|---|---|---|
| Hematology | | | | Change to | | | | | | Change to | | |
| Parameter | Screening | | | Day 42 | | | Screening | | | Day 42 | | |
| (Units) | N | Mean (SD) | | N | Mean (SD) | | N | Mean (SD) | | N | Mean (SD) | |
| Basophils (%) | 41 | 0.6 | (0.38) | 40 | 0.3 | (0.84) | 40 | 0.5 | (0.46) | 39 | 0.0 | (0.33) |
| Eosinophils | 41 | 3.0 | (1.82) | 40 | −0.4 | (1.56) | 40 | 2.3 | (1.24) | 39 | 0.3 | (1.53) |
| (%) | | | | | | | | | | | | |
| Hematocrit (%) | 41 | 38.4 | (3.15) | 40 | 3.7 | (2.96) | 40 | 38.4 | (3.33) | 39 | −0.8 | (5.32) |
| Hemoglobin | 41 | 12.3 | (1.21) | 40 | 1.2 | (0.88) | 40 | 12.3 | (1.19) | 39 | −0.1 | (0.92) |
| (g/dL) | | | | | | | | | | | | |
| Lymphocytes | 41 | 28.9 | (5.89) | 40 | −0.5 | (6.93) | 40 | 30.4 | (7.61) | 39 | −1.6 | (7.19) |
| (%) | | | | | | | | | | | | |
| MCH (pg) | 41 | 27.6 | (2.51) | 40 | 1.6 | (1.05) | 40 | 27.7 | (2.31) | 39 | −0.1 | (1.28) |
| MCHC (g/dL) | 41 | 32.1 | (1.22) | 40 | 0.2 | (1.56) | 40 | 32.0 | (1.11) | 39 | −0.3 | (1.23) |
| MCV (fL) | 41 | 85.9 | (6.28) | 40 | 4.6 | (5.00) | 40 | 86.6 | (6.46) | 39 | 0.6 | (3.63) |
| Monocytes (%) | 41 | 7.7 | (2.19) | 40 | 0.1 | (2.42) | 40 | 7.6 | (2.09) | 39 | 0.1 | (2.37) |
| Neutrophils | 41 | 59.9 | (7.33) | 40 | 0.5 | (8.60) | 40 | 59.3 | (8.24) | 39 | 1.0 | (8.39) |
| (%) | | | | | | | | | | | | |
| Platelets | 41 | 311.0 | (94.89) | 40 | −38.8 | (46.91) | 40 | 322.3 | (76.15) | 39 | −3.8 | (51.81) |
| (×10³/L) | | | | | | | | | | | | |
| RBC (×10⁶/L) | 41 | 4.5 | (0.36) | 40 | 0.2 | (0.23) | 40 | 4.5 | (0.51) | 39 | −0.1 | (0.35) |
| RDW (%) | 41 | 14.8 | (1.82) | 39 | 1.1 | (1.71) | 40 | 14.7 | (1.55) | 39 | −0.1 | (1.43) |
| Reticulocyte | 40 | 1.6 | (0.56) | 39 | 0.0 | (0.43) | 37 | 1.4 | (0.33) | 35 | 0.2 | (0.52) |
| Count (%) | | | | | | | | | | | | |
| WBC | 41 | 8.1 | (2.40) | 40 | −0.1 | (2.14) | 40 | 8.3 | (2.79) | 39 | −0.3 | (1.97) |

(×10³/μL)

Chemistry.

Mean increases from screening to Day 42 were observed for alkaline phosphatase, ALT, AST, and GGT in the Injectafer® group compared to mean decreases in the placebo group. A summary of mean changes from screening to Day 42 in chemistry parameters is presented in TABLE 18.

TABLE 18
Mean Changes From Screening to Day 42 in Chemistry Parameters (Safety Population)

| | Injectafer ® | | | | | | Placebo | | | | | |
|---|---|---|---|---|---|---|---|---|---|---|---|---|
| Chemistry | | | | Change to | | | | | | Change to | | |
| Parameters | Screening | | | Day 42 | | | Screening | | | Day 42 | | |
| (Units) | N | Mean (SD) | | N | Mean (SD) | | N | Mean (SD) | | N | Mean (SD) | |
| Albumin (g/dL) | 41 | 4.1 | (0.34) | 40 | 0.1 | (0.24) | 40 | 4.1 | (0.23) | 38 | −0.1 | (0.27) |
| Alkaline | 41 | 74.2 | (24.07) | 40 | 6.1 | (13.22) | 40 | 84.8 | (25.50) | 38 | −4.6 | (12.19) |
| Phosphatase | | | | | | | | | | | | |
| (U/L) | | | | | | | | | | | | |
| ALT (SGPT) | 41 | 26.6 | (14.81) | 40 | 4.2 | (13.74) | 40 | 25.0 | (12.75) | 38 | −0.2 | (9.62) |
| (U/L) | | | | | | | | | | | | |
| AST (SGOT) | 41 | 26.4 | (12.44) | 40 | 6.3 | (14.21) | 40 | 32.2 | (31.75) | 38 | −2.8 | (35.17) |
| (U/L) | | | | | | | | | | | | |
| Bicarbonate | 41 | 26.7 | (2.47) | 40 | −0.1 | (2.70) | 40 | 26.0 | (4.50) | 38 | 0.5 | (4.17) |
| (mmol/L) | | | | | | | | | | | | |
| BUN (mg/dL) | 41 | 13.3 | (4.73) | 40 | −0.7 | (4.54) | 40 | 12.4 | (3.91) | 38 | 0.7 | (3.94) |
| Calcium | 41 | 9.3 | (0.40) | 40 | 0.1 | (0.49) | 40 | 9.3 | (0.32) | 38 | 0.1 | (0.44) |
| (mg/dL) | | | | | | | | | | | | |
| Chloride | 41 | 104.1 | (2.68) | 40 | −0.9 | (3.06) | 40 | 103.2 | (2.53) | 38 | 0.4 | (2.79) |
| (mmol/L) | | | | | | | | | | | | |
| Creatinine | 41 | 0.8 | (0.15) | 40 | 0.0 | (0.17) | 40 | 0.7 | (0.11) | 38 | 0.0 | (0.13) |
| (mg/dL) | | | | | | | | | | | | |
| CRP (mg/dL) | 31 | 2.2 | (3.11) | 18 | −1.1 | (3.68) | 31 | 2.8 | (3.01) | 19 | −0.5 | (2.16) |
| Glucose | 41 | 92.3 | (17.04) | 40 | 0.3 | (22.28) | 40 | 98.2 | (28.39) | 38 | 6.6 | (31.96) |
| (mg/dL) | | | | | | | | | | | | |
| GGT (U/L) | 40 | 29.9 | (33.48) | 39 | 8.0 | (21.94) | 40 | 29.0 | (16.20) | 38 | −1.2 | (10.57) |
| LDH (U/L) | 41 | 329.7 | (158.98) | 40 | 29.9 | (79.25) | 40 | 343.5 | (178.85) | 38 | 11.7 | (62.42) |
| Magnesium | 41 | 2.0 | (0.22) | 40 | 0.0 | (0.18) | 40 | 1.9 | (0.20) | 38 | −0.0 | (0.20) |
| (mg/dL) | | | | | | | | | | | | |
| Phosphorus | 41 | 3.8 | (0.68) | 40 | −0.7 | (0.83) | 40 | 3.7 | (0.59) | 38 | −0.0 | (0.81) |
| (mg/dL) | | | | | | | | | | | | |
| Potassium | 41 | 4.4 | (0.44) | 40 | −0.3 | (0.49) | 40 | 4.2 | (0.50) | 38 | 0.1 | (0.60) |
| (mmol/L) | | | | | | | | | | | | |
| Sodium | 41 | 141.2 | (2.89) | 40 | 0.1 | (2.66) | 40 | 140.9 | (2.87) | 38 | 0.4 | (2.26) |
| (mmol/L) | | | | | | | | | | | | |
| Total Bilirubin | 36 | 0.4 | (0.24) | 34 | 0.1 | (0.23) | 32 | 0.4 | (0.16) | 30 | 0.0 | (0.14) |
| (mg/dL) | | | | | | | | | | | | |

Other Laboratory Assessments

A larger mean increase from screening to Day 42 in hepcidin was observed in the Injectafer® group than in the placebo group. A summary of mean changes from screening to Day 42 in other laboratory parameters is presented in TABLE 19.

TABLE 19
Mean Changes From Baseline to Day 35 in Other Laboratory Parameters (Safety Population)

| | Injectafer ® | | | | | | Placebo | | | | | |
|---|---|---|---|---|---|---|---|---|---|---|---|---|
| Chemistry | | | | Change to | | | | | | Change to | | |
| Parameters | Screening | | | Day 42 | | | Screening | | | Day 42 | | |
| (Units) | N | Mean (SD) | | N | Mean (SD) | | N | Mean (SD) | | N | Mean (SD) | |
| Absolute | 41 | 67.5 | (26.54) | 39 | 4.8 | (22.53) | 40 | 58.4 | (20.03) | 39 | 10.8 | (30.14) |
| reticulocytes | | | | | | | | | | | | |
| (k/μl) | | | | | | | | | | | | |
| Total protein | 24 | 7.3 | (0.46) | 23 | 0.1 | (0.58) | 22 | 10.2 | (12.91) | 22 | −2.9 | (12.94) |
| (g/dL) | | | | | | | | | | | | |
| Hepcidin | 37 | 27.9 | (18.07) | 36 | 105.8 | (57.62) | 36 | 24.4 | (16.66) | 33 | 0.4 | (13.81) |
| ng/mL) | | | | | | | | | | | | |
| MPV (FL) | 24 | 11.2 | (1.07) | 23 | 0.1 | (0.44) | 22 | 10.5 | (0.98) | 22 | 0.1 | (0.39) |

Individual Potentially Clinically Significant Abnormalities

Hematology.

No subjects were reported with treatment emergent PCS hematology values during the study.

Chemistry.

The only treatment-emergent PCS chemistry results were for low phosphorus in 3 subjects treated with Injectafer®. A PCS low phosphorus was defined as a baseline value that was within normal limits and decreased to a value defined as Grade 3 (<2.0 to 1.0 mg/dL) or Grade 4 (<1.0 mg/dL) per the CTCAE definitions. Details, including any treatment-emergent adverse event experienced, for the 3 subjects follow.
Subject 8001-105 had a baseline phosphorus value of 3.4 mg/dL (normal range: 2.5 to 5 mg/dL) and a Day 42 value of 1.9 mg/dL. The follow up phosphorus value on Day 259 was 4.6 mg/dL (normal range: 2.5 to 4.5 mg/dL). The subject was administered a total Injectafer® dose of 1500 mg elemental iron. The subject experienced a headache of Grade 1 severity on Day 0 that lasted 2.5 hours.
Subject 8001-113 had a baseline phosphorus value of 2.8 mg/dL (normal range: 2.5 to 5 mg/dL) and a Day 41 value of 1.9 mg/dL. The follow up phosphorus value on Day 69 was 2.6 mg/dL (normal range: 2.5 to 4.5 mg/dL). The subject was administered a total Injectafer® dose of 1500 mg elemental iron. The subject experienced dermatitis of Grade 1 severity that started on Day 18 and resolved on Day 29.
Subject 8001-173 had a baseline phosphorus value of 3.5 mg/dL (normal range: 2.5 to 4.5 mg/dL) and a Day 41 value of 1.4 mg/dL. The follow up phosphorus value on Day 137 was 3.9 mg/dL (normal range: 2.5 to 4.5 mg/dL). The subject was administered a total Injectafer® dose of 1500 mg elemental iron. No adverse events were reported for the subject.

Vital Signs, Physical Findings, and Other Observations Related to Safety

Vital Signs.

No clinically important treatment group differences for mean changes in systolic and diastolic blood pressure were noted. One (2.4%) subject in the Injectafer® group had a treatment-emergent PCS low systolic blood pressure (≤90 mmHg with ≥20 mmHg decrease from pre-dose) immediately post-dose on Day 0 and 1 (2.6%) subject in the placebo group had a treatment-emergent PCS low systolic blood pressure immediately post-dose on Day 5.
The sitting systolic/diastolic blood pressure for Subject 8001-158 (Injectafer®) was 116/78 mmHg at pre-dose, 85/70 mmHg at immediately post-dose, and 133/98 mmHg at 30 minutes post-dose on Day 0. The subject was administered 750 mg Injectafer® on Day 5. No adverse events were reported for the subject.
The sitting systolic/diastolic blood pressure for Subject 8001-162 (placebo) was 127/82 mmHg at pre-dose, 88/51 mmHg at immediately post-dose, and 108/67 mmHg at 30 minutes post-dose on Day 5. No adverse events were reported for the subject.

One (1; 2.4%) subject in the Injectafer® group had a treatment-emergent PCS high pulse immediately post-dose (≥100 bpm with ≥5 bpm increase from pre-dose) on Day 5, and 1 (2.4%) subject in the placebo group had a treatment emergent PCS low pulse (≤50 bpm with ≥5 bpm decrease from pre-dose) immediately post-dose on Day 5.
The sitting pulse for Subject 8001-112 (Injectafer®) was 101 bpm at pre-dose, 120 bpm immediately post-dose, and 100 bpm at 30 minutes post-dose on Day 5. The subject was administered 750 mg Injectafer® on Day 5. No adverse events were reported for the subject.
The sitting pulse for Subject 8001-162 (placebo) was 64 bpm at pre-dose, 48 bpm immediately post-dose, and 69 bpm at 30 minutes post-dose on Day 5. No adverse events were reported for the subject.

Physical Examination

Clinically significant deteriorations in physical examination findings were reported as treatment emergent adverse events, which are summarized above.

Safety Conclusions During the study, at least 1 treatment emergent adverse event was experienced by 29.3% of the subjects in the Injectafer® group and 5.0% of the subjects in the placebo group. Treatment emergent adverse events reported by ≥2 subjects in the Injectafer® group were nausea (7.3%), flushing (14.6%), and dizziness (4.9%). No treatment emergent adverse events were reported by ≥2 subjects in the placebo group. All treatment-emergent adverse events were mild in severity.

At least 1 drug related treatment emergent adverse event (defined as possibly or probably related) was experienced by 24.4% of the subjects in the Injectafer® group and no subjects in the placebo group. Drug related treatment emergent adverse events experienced by ≥2 subjects in the Injectafer® group were flushing (12.2%), dizziness (4.9%), and nausea (4.9%).

No subjects died, experienced treatment emergent serious adverse events, or discontinued study drug due to treatment emergent adverse events during the study.

Mean increases from screening to Day 42 were observed for hematocrit, hemoglobin, MCH, MCHC, RBC, and RDW in the Injectafer® group compared to mean decreases in the placebo group. A greater mean increase in MCV from screening to Day 42 was observed for subjects in the Injectafer® group compared to subjects in the placebo group. A greater mean decrease in platelets from screening to Day 42 was observed for subjects in the Injectafer® group compared to subjects in the placebo group. No subjects were reported with treatment emergent PCS hematology values during the study.

Mean increases from screening to Day 42 were observed for alkaline phosphatase, ALT, AST, and GGT in the Injectafer® group compared to mean decreases in the placebo group. The only treatment-emergent PCS chemistry results were for low phosphorus in 3 subjects treated with Injectafer®. The lowest observed phosphorous value was 1.4 mg/dL. The subjects were asymptomatic and phosphorus values returned to within the normal range.

A larger mean increase from screening to Day 42 in hepcidin was observed in the Injectafer® group than in the placebo group.

There were no clinically important trends in vital signs.

In summary, Injectafer® was safe and well-tolerated in this study.

### Example 12: Overall Conclusions

The following example describes the overall conclusions of the study.

The primary efficacy endpoint was the percentage of subjects who had a ≥3-point improvement in the FIQR from baseline to Day 42. The proportion of subjects achieving the primary endpoint was greater in the Injectafer® group than the placebo group (approximately 77% versus 67%). Compared to the responder rates specified for sample size calculation, the rate for Injectafer® was near the pre-specified 60% to 75% rate, whereas the placebo rate of 67% was more than twice the pre-specified 30% rate. Thus, the study had insufficient power to detect the clinically important treatment group difference of approximately 10 percentage points. However, statistically significantly greater mean improvement from baseline in FIQR total score was observed for the Injectafer® group as compared to the placebo on Days 14, 28, and 42.

Corroborating evidence for the efficacy of Injectafer® included the following:
Statistically significantly greater improvement from baseline in both the Brief Pain Inventory Pain Severity and Pain Interference scores was observed for the Injectafer® group as compared to the placebo group on Days 14, 28, and 42.
Statistically significantly greater improvement from baseline in the Fatigue Visual Numeric Scale was observed for the Injectafer® group as compared to the placebo group on Days 14, 28, and 42.
Statistically significantly greater mean increases from baseline to Day 42 in serum ferritin, iron, and TSAT were observed for the Injectafer® group as compared to the placebo. A statistically significantly greater mean decrease from baseline to Day 42 in TIBC was observed for the Injectafer® group as compared to the placebo group.

In this study, the mean change in hemoglobin from baseline to Day 42 was 1.2 g/dL in the Injectafer group and −0.1 g/dL in the placebo group. The mean hemoglobin value at baseline was 12.3 g/dL in both treatment groups.

During the study, at least 1 treatment emergent adverse event was experienced by 29.3% of the subjects in the Injectafer® group and 5.0% of the subjects in the placebo group. Treatment emergent adverse events reported by ≥2 subjects in the Injectafer® group were nausea (7.3%), flushing (14.6%), and dizziness (4.9%). No treatment emergent adverse events were reported by ≥2 subjects in the placebo group. All treatment-emergent adverse events were mild in severity. Drug related treatment emergent adverse events experienced by ≥2 subjects in the Injectafer® group were flushing (12.2%), dizziness (4.9%), and nausea (4.9%).

No subjects died, experienced treatment emergent serious adverse events, or discontinued study drug due to treatment emergent adverse events during the study.

Mean increases from screening to Day 42 were observed for hematocrit, MCH, MCHC, RBC, and RDW in the Injectafer® group compared to mean decreases in the placebo group. A greater mean increase in MCV from screening to Day 42 was observed for subjects in the Injectafer® group compared to subjects in the placebo group. These differences are consistent with the increase in iron indices due to the efficacy of Injectafer®.

No subjects were reported with treatment emergent PCS hematology values during the study. The only treatment-emergent PCS chemistry results were for low phosphorus in 3 subjects treated with Injectafer®. The subjects were asymptomatic and values returned to within the normal range. The decrease in phosphorous is a known, transient effect of Injectafer®. There were no clinically important trends in vital signs.

In conclusion, statistically significantly greater mean improvement was observed for Injectafer® as compared to placebo for FIQR total score, Brief Pain Inventory Pain Severity and Pain Interference scores, Fatigue Visual Numeric Scale, and iron indices. Injectafer® was safe and well tolerated in this study. The study provided proof-of-concept for the treatment of fibromyalgia with Injectafer®.

### REFERENCES

Atkinson M J, Kumar R, Cappelleri J C, Hass S L. Hierarchical construct validity of the treatment satisfaction questionnaire for medication (TSQM version II) among outpatient pharmacy consumers. Value Health. 2005 November-December; 8 Suppl 1:S9-S24
Pamuk G E et al. “An increased prevalence of fibromyalgia in iron deficiency anemia and thalassemia minor and assoicated factors.” Clin Rheumatol. 2008; 27(9) 1103-8.
Qunibi W Y. The efficacy and safety of current intravenous iron preparations for the management of iron-deficiency anaemia: a review. Arzneimittelforschung. 2010; 60:399-412.
Gerwin, Robert. “A Review of Myofascial pain and Fibromyalgia—Factors that Promote their Persistence” Acupuncture in Medicine. 2005; 23(3): 121-134.
Geisser, Peter et al “The Pharmacokinetics and Pharmacodynamics of Iron Preparations” Pharmaceutics 2011, 3, 12-33
Fishbane S, et al. The safety of intravenous iron dextran in hemodialysis patients. Am J Kidney Dis. 1996; 28:529-534.
Wysowski D K et al. Use of parenteral iron products and serious anaphylactic-type reactions. Am J Hematol. 2010; 85:650-654.

1. A method of treating Fibromyalgia (FM) or Fibromyalgia syndrome (FMS), comprising:
administering an iron carbohydrate complex to a subject in need thereof, wherein the iron carbohydrate complex is selected from among an iron carboxymaltose complex, iron sucrose complex, an iron mannitol complex, an iron polyisomaltose complex, an iron polymaltose complex, an iron gluconate complex, an iron sorbitol complex, an iron polyglucose sorbitol carboxymethyl ether complex, an iron polyglucose sorbitol, iron carboxymethyl ether complex, and an iron hydrogenated dextran complex.

2. The method of claim 1, wherein the iron carbohydrate complex has a substantially non-immunogenic carbohydrate component.

3. The method of claim 1, wherein the iron carbohydrate complex is selected from among an iron carboxymaltose complex, an iron polyglucose sorbitol carboxymethyl ether complex and an iron polyisomaltose complex.

4. The method of claim 3, wherein the iron carbohydrate complex is a carboxymaltose complex that comprises polynuclear iron (III)-hydroxide 4(R)-(poly-(1→4)-O-α-glucopyranosyl)-oxy-2(R),3(S),5(R),6-tetrahydroxy-hexanoate (VIT-45).

5. The method of claim 3, wherein the iron carbohydrate complex is an iron polyglucose sorbitol carboxymethyl ether complex that comprises Ferumoxytol.

6. The method of claim 3, wherein the iron carbohydrate complex is iron polyisomaltose that comprises iron isomaltoside 1000.

7. The method of claim 1, wherein administration of the iron carbohydrate complex is administered in an amount effective to inhibit, slow, limit, remove, or prevent symptoms associated with Fibromyalgia (FM) or Fibromyalgia syndrome (FMS).

8. The method of claim 1, wherein administration of the iron carbohydrate complex substantially inhibits, slows, limits, removes, or prevents one or more of: chronic widespread pain, painful response to pressure, painful response to tactile pressure (allodynia), fatigue, headache, debilitating fatigue, sleep disturbance, joint stiffness, morning stiffness, difficulty with swallowing, bowel abnormalities, bladder abnormalities, numbness, tingling, tingling of the skin (paresthesias), prolonged muscle spasms, weakness in the limbs, nerve pain, muscle twitching, palpitations, functional bowel disturbances, irritable bowel syndrome (IBS), cognitive dysfunction, depression, anxiety, stress-related disorders, interstitial cystitis (IC), dyspareunia, or mood disturbance.

9. The method of claim 1, wherein administration of the iron carbohydrate complex is in an amount that substantially reduces Revised Fibromyalgia Impact Questionnaire (FIQR) value, reduces International Restless Legs Syndrome (IRLS) value, reduces Brief Pain Inventory (BPI) value, reduces Pain Severity value, reduces Pain Interference value, reduces Fatigue Visual Numeric value, reduces required intervention for fibromyalgia, reduces an amount of time to fibromyalgia intervention, or reduces proportion of relapse, or any combination thereof when compared to baseline values.

10. The method of claim 1, wherein the iron carbohydrate complex is administered in a single dosage unit of at least about 0.1 grams of elemental iron.

11. The method of claim 1, wherein the iron carbohydrate complex is administered in a single dosage unit of at least about 0.1 grams, at least about 0.2 grams, at least about 0.3 grams, at least about 0.4 grams, at least about 0.5 grams, 0.6 grams, at least about 0.7 grams; at least about 0.8 grams; at least about 0.9 grams; at least about 1.0 grams; at least about 1.1 grams; at least about 1.2 grams; at least about 1.3 grams; at least about 1.4 grams; at least about 1.5 grams; at least about 1.6 grams; at least about 1.7 grams; at least about 1.8 grams; at least about 1.9 grams; at least about 2.0 grams; at least about 2.1 grams; at least about 2.2 grams; at least about 2.3 grams; at least about 2.4 grams; or at least about 2.5 grams of elemental iron.

12. (canceled)

13. The method of claim 1, wherein the iron carbohydrate complex is administered at a single dosage unit of up to about 0.1 grams, up to about 0.2 grams, up to about 0.3 grams, up to about 0.4 grams, up to about 0.5 grams, 0.6 grams, up to about 0.7 grams; up to about 0.8 grams; up to about 0.9 grams; up to about 1.0 grams; up to about 1.1 grams; up to about 1.2 grams; up to about 1.3 grams; up to about 1.4 grams; up to about 1.5 grams; up to about 1.6 grams; up to about 1.7 grams; up to about 1.8 grams; up to about 1.9 grams; up to about 2.0 grams; up to about 2.1 grams; up to about 2.2 grams; up to about 2.3 grams; up to about 2.4 grams; or up to about 2.5 grams of elemental iron.

14. The method of claim 1, wherein the iron carbohydrate complex is administered in about 15 minutes or less.

15. The method of claim 1, wherein the iron carbohydrate complex is administered in about 14 minutes or less, about 13 minutes or less, about 12 minutes or less, about 11 minutes or less, about 10 minutes or less, about 9 minutes or less, about 8 minutes or less, about 7 minutes or less, about 6 minutes or less, about 5 minutes or less, about 4 minutes or less, about 3 minutes or less, or about 2 minutes or less.

16. The method of claim 1, further comprising administering pregablin, duloxetine, or milnacipran.

17. The method of claim 1, wherein the amount of time after administration of the iron carbohydrate complex in which symptoms associated with Fibromyalgia (FM) or Fibromyalgia syndrome (FMS) are substantially inhibited, slowed, limited, removed, or prevented is between about 0 days and about 50 days.

18. The method of claim 1, wherein the amount of time after administration of the iron carbohydrate complex in which symptoms associated with Fibromyalgia (FM) or Fibromyalgia syndrome (FMS) are substantially inhibited, slowed, limited, removed, or prevented is about 14 days, about 28 days, or about 42 days.

19. The method of claim 3, wherein the iron carbohydrate complex is iron polyisomaltose that comprises a linear chemical structure of repeating α-1,6 linked glucose units.

20. The method of claim 3, wherein the iron carbohydrate complex is iron polyisomaltose that is a linear chemical structure of repeating α-1,6 linked glucose units.
